SEQUENCE LISTING (217 sequences)

SEQ ID NO: 1 - DNA, 26 bp, Acidobacteria bacterium Ellin345
ttgacctcaagtttacttgaggtttt

SEQ ID NO: 2 - DNA, 26 bp, Acidovorax avenae subsp. citrulli AAC00-1
ttgacctcaagtttagttgaggttcc

SEQ ID NO: 3 - DNA, 26 bp, Acidovorax avenae subsp. citrulli AAC00-1
ttgacctcaacaatggttgaggtttg

SEQ ID NO: 4 - DNA, 26 bp, Acinetobacter baumannii ATCC 17978
ttgacctcaacttaagttgaggttgg

SEQ ID NO: 5 - DNA, 26 bp, Acinetobacter baumannii AYE
ttgacctcaacttaagttgaggttgg

SEQ ID NO: 6 - DNA, 26 bp, Acinetobacter baumannii AYE
ttgacttcaagtgaactttaacttgc

SEQ ID NO: 7 - DNA, 26 bp, Acinetobacter baumannii (SDF)
ttgacctcaacttaagttgaggttgg

SEQ ID NO: 8 - DNA, 26 bp, Acinetobacter sp. ADP1
ttgacctcaagttaacttgagctttg

SEQ ID NO: 9 - DNA, 26 bp, Aeromonas hydrophila subsp. hydrophila ATCC 7966
ttgacctcaagttagctttaacttgc

SEQ ID NO: 10 - DNA, 26 bp, Aeromonas salmonicida subsp. salmonicida A449
ttgacctcaagttagctttaacttgc

SEQ ID NO: 11 - DNA, 26 bp, Agrobacterium tumefaciens str. C58 chromosome circular
ttgacctcaactatggttgaggaatt

SEQ ID NO: 12 - DNA, 26 bp, Agrobacterium tumefaciens str. C58 chromosome linear
ttgacctcaactatagttgaggaatt

SEQ ID NO: 13 - DNA, 26 bp, Agrobacterium tumefaciens str. C58 chromosome linear
tcgacctcaactcaagttgaggttgt

SEQ ID NO: 14 - DNA, 26 bp, Agrobacterium tumefaciens str. C58 plasmid At
ttgacctcaactctagttgaggttgt

SEQ ID NO: 15 - DNA, 26 bp, Arthrobacter aurescens TC1
ttgacctcaacttaagttgaggtcct

SEQ ID NO: 16 - DNA, 26 bp, Azoarcus sp. BH72
ttgacctcaagtcgacttgaacttgc

SEQ ID NO: 17 - DNA, 26 bp, Bacillus anthracis str. ′Ames Ancestor′
ttgacctaaacttaagtttaggctgt

SEQ ID NO: 18 - DNA, 26 bp, Bacillus anthracis str. Ames
ttgacctaaacttaagtttaggctgt

SEQ ID NO: 19 - DNA, 26 bp, Bacillus anthracis str. Sterne
ttgacctaaacttaagtttaggctgt

SEQ ID NO: 20 - DNA, 26 bp, Bacillus cereus ATCC 14579
ttgacctaaacttaagtttaggctgt

SEQ ID NO: 21 - DNA, 26 bp, Bacillus cereus E33L
ttgacctaaacttaagtttaggctgt

SEQ ID NO: 22 - DNA, 26 bp, Bacillus subtilis subsp. subtilis str. 168
ttgacttaaagttaactttaagtgtt

SEQ ID NO: 23 - DNA, 26 bp, Bacillus thuringiensis str. Al Hakam
ttgacctaaacttaagtttaggctgt

SEQ ID NO: 24 - DNA, 26 bp, Bacillus thuringiensis serovar konkukian str. 97-27
ttgacctaaacttaagtttaggctgt

SEQ ID NO: 25 - DNA, 26 bp, Bdellovibrio bacteriovorus HD100
ttgacctcaagttaacttgaggttgt

SEQ ID NO: 26 - DNA, 26 bp, Bordetella bronchiseptica RB50
ttgacctcaagctagcttgagggtcc

SEQ ID NO: 27 - DNA, 26 bp, Bordetella pertussis Tohama I
ttgacctcaagctagcttgagggtcc

SEQ ID NO: 28 - DNA, 26 bp, Burkholderia sp. 383 chromosome 2
ttgacctcaagtgaacttgaagttgc

SEQ ID NO: 29 - DNA, 26 bp, Burkholderia sp. 383 chromosome 2
ttgacttgaagttaacttcaactttt

SEQ ID NO: 30 - DNA, 26 bp, Burkholderia ambifaria MC40-6
ttgacctcaagtgaacttgaagttgc

SEQ ID NO: 31 - DNA, 26 bp, Burkholderia ambifaria MC40-6
ttgacttgaagttaacttcaactttt

SEQ ID NO: 32 - DNA, 26 bp, Burkholderia cenocepacia AU 1054 chromosome 2
ttgacctcaagtgaacttgaagttgc

SEQ ID NO: 33 - DNA, 26 bp, Burkholderia cenocepacia AU 1054 chromosome 2
ttgacttgaagttaacttcaactttt

SEQ ID NO: 34 - DNA, 26 bp, Burkholderia cenocepacia HI2424 chromosome 2
ttgacctcaagtgaacttgaagttgc

SEQ ID NO: 35 - DNA, 26 bp, Burkholderia cenocepacia HI2424 chromosome 2
ttgacttgaagttaacttcaactttt

SEQ ID NO: 36 - DNA, 26 bp, Burkholderia cenocepacia MC0-3
ttgacctcaagtgaacttgaagttgc

SEQ ID NO: 37 - DNA, 26 bp, Burkholderia cenocepacia MC0-3
ttgacttgaagttaacttcaactttt

SEQ ID NO: 38 - DNA, 26 bp, Burkholderia cepacia AMMD chromosome 2
ttgacctcaagtgaacttgaagttgc

SEQ ID NO: 39 - DNA, 26 bp, Burkholderia cepacia AMMD chromosome 2
ttgacttgaagttaacttcaactttt

SEQ ID NO: 40 - DNA, 26 bp, Burkholderia multivorans ATCC 17616
ttgacctcaagtgagcttgaagttgc

SEQ ID NO: 41 - DNA, 26 bp, Burkholderia multivorans ATCC 17616
ttgacttgaagttaacttgaactttt

SEQ ID NO: 42 - DNA, 26 bp, Chromobacterium violaceum ATCC 12472
ttgacttcaagttaacttgaactttg

SEQ ID NO: 43 - DNA, 26 bp, Chromobacterium violaceum ATCC 12472
ttgacttcaagttaactttaactttc

SEQ ID NO: 44 - DNA, 26 bp, Citrobacter koseri ATCC BAA-895
tttacctcaagttaacttgaggaatt

SEQ ID NO: 45 - DNA, 26 bp, Clavibacter michiganensis subsp. Sepedonicus
ttgacctcaagttcgcttgaggtgct

SEQ ID NO: 46 - DNA, 26 bp, Clostridium beijerinckii NCIMB 8052
ttgacctaaagttaactttagggtgt

SEQ ID NO: 47 - DNA, 26 bp, Clostridium beijerinckii NCIMB 8052
ttgacttaaagttaactttaagtgtt

SEQ ID NO: 48 - DNA, 26 bp, Delftia acidovorans SPH-1
ttgacctcaactttggttgaggtttc

SEQ ID NO: 49 - DNA, 26 bp, Desulfitobacterium hafniense Y51
ttgacctaaagttaagtttaggtgct

SEQ ID NO: 50 - DNA, 26 bp, Enterobacter sp. 638
ttgacctcaagttaactttagctttt

SEQ ID NO: 51 - DNA, 26 bp, Enterobacter sp. 638
tttacctcaagttaacttgaggaatt

SEQ ID NO: 52 - DNA, 26 bp, Enterobacter sakazakii ATCC BAA-894
tttacctcaagttaacttgaggaatt

SEQ ID NO: 53 - DNA, 26 bp, Erythrobacter litoralis HTCC2594
ttgacctcaagtcagcttgaggttgc

SEQ ID NO: 54 - DNA, 26 bp, Escherichia coli 536
tttacctcaagttaacttgaggaatt

SEQ ID NO: 55 - DNA, 26 bp, Escherichia coli APEC O1
tttacctcaagttaacttgaggaatt

SEQ ID NO: 56 - DNA, 26 bp, Escherichia coli CFT073
tttacctcaagttaacttgaggaatt

SEQ ID NO: 57 - DNA, 26 bp, Escherichia coli DH10B
tttacctcaagttaacttgaggaatt

SEQ ID NO: 58 - DNA, 26 bp, Escherichia coli E24377A
tttacctcaagttaacttgaggaatt

SEQ ID NO: 59 - DNA, 26 bp, Escherichia coli HS
tttacctcaagttaacttgaggaatt

SEQ ID NO: 60 - DNA, 26 bp, Escherichia coli str. K-12 substr. MG1655
tttacctcaagttaacttgaggaatt

SEQ ID NO: 61 - DNA, 26 bp, Escherichia coli O157:H7 EDL933
tttacctcaagttaacttgaggaatt

SEQ ID NO: 62 - DNA, 26 bp, Escherichia coli O157:H7 str. Sakai
tttacctcaagttaacttgaggaatt

SEQ ID NO: 63 - DNA, 26 bp, Escherichia coli SECEC SMS-3-5
tttacctcaagttaacttgaggaatt

SEQ ID NO: 64 - DNA, 26 bp, Escherichia coli UTI89
tttacctcaagttaacttgaggaatt

SEQ ID NO: 65 - DNA, 26 bp, Frankia sp. CcI3
ttgacttcaatcatggttgaggtttt

SEQ ID NO: 66 - DNA, 26 bp, Frankia sp. EAN1pec
ttgacctgaagtcaacttcagatatt

SEQ ID NO: 67 - DNA, 26 bp, Frankia sp. EAN1pec
ttgacctgaagtcaggtttaactttt

SEQ ID NO: 68 - DNA, 26 bp, Hahella chejuensis KCTC 2396
ttgacctaaagttaagttgaggtttt

SEQ ID NO: 69 - DNA, 26 bp, Hahella chejuensis KCTC 2396
ttgacctcaagtcgacttgagcttgt

SEQ ID NO: 70 - DNA, 26 bp, Herpetosiphon aurantiacus ATCC 23779
ttgacttccagttaacttgaacttgt

SEQ ID NO: 71 - DNA, 26 bp, Hyphomonas neptunium ATCC 15444
ttgatctaaagtgaacttgagattgt

SEQ ID NO: 72 - DNA, 26 bp, Idiomarina loihiensis L2TR
ttgacctaaagttaactttaagttgt

SEQ ID NO: 73 - DNA, 26 bp, Janthinobacterium sp. Marseille
ttgacctcaagttaagttgaggtttt

SEQ ID NO: 74 - DNA, 26 bp, Kineococcus radiotolerans SRS30216
ttgacctcaagtccgcttgagcaggt

SEQ ID NO: 75 - DNA, 26 bp, Klebsiella pneumoniae subsp. pneumoniae MGH 78578
tttacctcaagttaacttgaggaatt

SEQ ID NO: 76 - DNA, 26 bp, Klebsiella pneumoniae subsp. pneumoniae MGH 78578
ttgatctcaagttaacttgaggttgt

SEQ ID NO: 77 - DNA, 26 bp, Lactobacillus casei ATCC 334
ttgacctaaagctaactttaggggtt

SEQ ID NO: 78 - DNA, 26 bp, Lactobacillus sakei subsp. sakei 23K
ttgacctcaagtcagcttgaggttgt

SEQ ID NO: 79 - DNA, 26 bp, Lactobacillus salivarius UCC118
ttgacttcaagttaacttgaagtttt

SEQ ID NO: 80 - DNA, 26 bp, Lactococcus lactis subsp. cremoris MG1363
ttgctctcaagttaacttgagggttt

SEQ ID NO: 81 - DNA, 26 bp, Lactococcus lactis subsp. cremoris SK11
ttgctctcaagttaacttgagggttt

SEQ ID NO: 82 - DNA, 26 bp, Lactococcus lactis subsp. lactis Il1403
ttgccctcaagttaacttgaaggttt

SEQ ID NO: 83 - DNA, 26 bp, Maricaulis maris MCS10
ttgacctcaaccaaggttaagcaatt

SEQ ID NO: 84 - DNA, 26 bp, Marinomonas sp. MWYL1
ttgacctcaacttaagttcaggttgc

SEQ ID NO: 85 - DNA, 26 bp, Mesorhizobium loti MAFF303099
ttgacctcaacttaagttgagattgt

SEQ ID NO: 86 - DNA, 26 bp, Mesorhizobium loti MAFF303099
ttgacctcaagttatgttgagcttgt

SEQ ID NO: 87 - DNA, 26 bp, Mycobacterium abscessus
ttgacctcaagtccagttgaggattt

SEQ ID NO: 88 - DNA, 26 bp, Mycobacterium abscessus
ttgacctcaagtgcgcttgagatttt

SEQ ID NO: 89 - DNA, 26 bp, Mycobacterium gilvum PYR-GCK
ttgacctgaaggttggttgaggttgc

SEQ ID NO: 90 - DNA, 26 bp, Mycobacterium sp. JLS
ttgacctgaactttggttgaggtctt

SEQ ID NO: 91 - DNA, 26 bp, Mycobacterium sp. JLS
ttgacctgaactttggttgaggtcgg

SEQ ID NO: 92 - DNA, 26 bp, Mycobacterium sp. KMS
ttgacctgaactttggttgaggtctt

SEQ ID NO: 93 - DNA, 26 bp, Mycobacterium sp. KMS
ttgacctgaactttggttgaggtcgg

SEQ ID NO: 94 - DNA, 26 bp, Mycobacterium sp. MCS
ttgacctgaactttggttgaggtctt

SEQ ID NO: 95 - DNA, 26 bp, Mycobacterium sp. MCS
ttgacctgaactttggttgaggtcgg

SEQ ID NO: 96 - DNA, 26 bp, Mycobacterium smegmatis str. MC2 155
ttcacctgaagtaaggtttaggtgtc

SEQ ID NO: 97 - DNA, 26 bp, Mycobacterium smegmatis str. MC2 155
ttgacctcaacctcacttgaggtgcc

SEQ ID NO: 98 - DNA, 26 bp, Mycobacterium smegmatis str. MC2 155
ttgacctcatcattggttgaggtttt

SEQ ID NO: 99 - DNA, 26 bp, Mycoplasma penetrans HF-2
ttgacttaaacttaggtttaagttgt

SEQ ID NO: 100 - DNA, 26 bp, Myxococcus xanthus DK 1622
ttgacctcaagtcgacttgaactggc

SEQ ID NO: 101 - DNA, 26 bp, Nocardia farcinica IFM 10152
ttgacttcaagtgaacttgaaatttt

SEQ ID NO: 102 - DNA, 26 bp, Nocardia farcinica IFM 10152
ttgacctcaacattggttgaggaagc

SEQ ID NO: 103 - DNA, 26 bp, Novosphingobium aromaticivorans DSM 12444
ttgatctcaagctaacttgaggttgc

SEQ ID NO: 104 - DNA, 26 bp, Ochrobactrum anthropi ATCC 49188 chromosome 2
ttgacctcaaccaaggttgaggaact

SEQ ID NO: 105 - DNA, 26 bp, Pelotomaculum thermopropionicum SI
tttacctcaagtcaacttaaggtgga

SEQ ID NO: 106 - DNA, 26 bp, Photobacterium profundum SS9 chromosome 2
ttgacctcaagttaacctgaggcact

SEQ ID NO: 107 - DNA, 26 bp, Polaromonas sp. JS666
ttgacctcaactttacttgaggtttt

SEQ ID NO: 108 - DNA, 26 bp, Pseudoalteromonas atlantica T6c
ttgatctaaagttagctttagatatt

SEQ ID NO: 109 - DNA, 26 bp, Pseudomonas aeruginosa PAO1
ttgacctcaacttaacttgaggtttt

SEQ ID NO: 110 - DNA, 26 bp, Pseudomonas aeruginosa PAO1
ttgacctcaagtttgcttgaggtttt

SEQ ID NO: 111 - DNA, 26 bp, Pseudomonas aeruginosa PAO1
tttacctcaagttaacttgagctatc

SEQ ID NO: 112 - DNA, 26 bp, Pseudomonas aeruginosa PA7
ttgacctcaagtttacttgaggtttt

SEQ ID NO: 113 - DNA, 26 bp, Pseudomonas aeruginosa PA7
tttacctcaagttaagttgagctatc

SEQ ID NO: 114 - DNA, 26 bp, Pseudomonas aeruginosa PA7
ttgacctcaacttaagttgaggttct

SEQ ID NO: 115 - DNA, 26 bp, Pseudomonas aeruginosa UCBPP-PA14
ttgacctcaacttaacttgaggtttt

SEQ ID NO: 116 - DNA, 26 bp, Pseudomonas aeruginosa UCBPP-PA14
ttgacctcaagtttgcttgaggtttt

SEQ ID NO: 117 - DNA, 26 bp, Pseudomonas aeruginosa UCBPP-PA14
tttacctcaagttaacttgagctatc

SEQ ID NO: 118 - DNA, 26 bp, Pseudomonas entomophila L48
ttgaccttgagttaactggaggtttt

SEQ ID NO: 119 - DNA, 26 bp, Pseudomonas fluorescens Pf-5
ttgaccttgacttaactagaggtttt

SEQ ID NO: 120 - DNA, 26 bp, Pseudomonas fluorescens PfO-1
tttacctctagttaactcgaggtttt

SEQ ID NO: 121 - DNA, 26 bp, Pseudomonas mendocina ymp
ttgaccttaagttaagttgaggtttt

SEQ ID NO: 122 - DNA, 26 bp, Pseudomonas putida F1
ttgacctcaagtaaagttgagctttt

SEQ ID NO: 123 - DNA, 26 bp, Pseudomonas putida F1
ttgacctcgagttaagtcaaggtttt

SEQ ID NO: 124 - DNA, 26 bp, Pseudomonas putida F1
ttgaccttgactttgcttgaggtttt

SEQ ID NO: 125 - DNA, 26 bp, Pseudomonas putida F1
ttgacctcgagttagcttaaggtctg

SEQ ID NO: 126 - DNA, 26 bp, Pseudomonas putida GB-1
ttgacctcaagttaagttgagctctt

SEQ ID NO: 127 - DNA, 26 bp, Pseudomonas putida GB-1
ttgaccttgactttgcttgaggtttt

SEQ ID NO: 128 - DNA, 26 bp, Pseudomonas putida KT2440
tcgacctaaagttaagttgagctttt

SEQ ID NO: 129 - DNA, 26 bp, Pseudomonas putida KT2440
ttgacctcgagttaagtcaaggtttt

SEQ ID NO: 130 - DNA, 26 bp, Pseudomonas putida W619
ttgaccttgagttaactggaggtttt

SEQ ID NO: 131 - DNA, 26 bp, Pseudomonas stutzeri A1501
ttgacctcaagttcgcttgaacttct

SEQ ID NO: 132 - DNA, 26 bp, Ralstonia eutropha H16 chromosome 1
ttgacctcaagtcgacttgaactggc

SEQ ID NO: 133 - DNA, 26 bp, Ralstonia eutropha H16 chromosome 2
ttgacctcaatttaggttgaggtttg

SEQ ID NO: 134 - DNA, 26 bp, Ralstonia metallidurans CH34 chromosome 2
ttgacctcaagcgtgcttgaggtttg

SEQ ID NO: 135 - DNA, 26 bp, Renibacterium salmoninarum ATCC 33209
ttgacctcaagttagcttgaggttct

SEQ ID NO: 136 - DNA, 26 bp, Renibacterium salmoninarum ATCC 33209
ttgacctcaacaaaggttgagggatt

SEQ ID NO: 137 - DNA, 26 bp, Rhizobium etli CFN 42
ttgacctcaaccatagttgaggaatt

SEQ ID NO: 138 - DNA, 26 bp, Rhizobium etli CFN 42
ttgacctcaatattagttgaggtttt

SEQ ID NO: 139 - DNA, 26 bp, Rhizobium etli CFN 42 plasmid p42f
ttaacttcaagttaacttgaacttgt

SEQ ID NO: 140 - DNA, 26 bp, Rhizobium leguminosarum bv. viciae 3841
ttgacctcaggttaacttgaggaatt

SEQ ID NO: 141 - DNA, 26 bp, Rhizobium leguminosarum bv. viciae 3841
ttgacctcaagattagttgaggtttt

SEQ ID NO: 142 - DNA, 26 bp, Rhodopseudomonas palustris BisB5
ttgacctcaactaaggttgaggttct

SEQ ID NO: 143 - DNA, 26 bp, Saccharopolyspora erythraea NRRL 2338
ttgacctcgaccttgcttgagctttt

SEQ ID NO: 144 - DNA, 26 bp, Saccharopolyspora erythraea NRRL 2338
ttgacctccagccaactcgaagtttc

SEQ ID NO: 145 - DNA, 26 bp, Salinispora tropica CNB-440, complete genome
ttgacctcaaccacggttgaggtttt

SEQ ID NO: 146 - DNA, 26 bp, Salinispora tropica CNB-440
ttgatctgaagttaacttcaggttgt

SEQ ID NO: 147 - DNA, 26 bp, Salmonella enterica subsp. arizonae serovar 62:z4,z23:--
tttacctcaagttaacttgaggaatt

SEQ ID NO: 148 - DNA, 26 bp, Salmonella enterica subsp. enterica serovar Choleraesuis str. SC-B67
tttacctcaagttaacttgaggaatt

SEQ ID NO: 149 - DNA, 26 bp, Salmonella enterica subsp. enterica serovar Paratyphi A str. ATCC 9150
tttacctcaagttaacttgaggaatt

SEQ ID NO: 150 - DNA, 26 bp, Salmonella enterica subsp. enterica serovar Paratyphi B str. SPB7
tttacctcaagttaacttgaggaatt

SEQ ID NO: 151 - DNA, 26 bp, Salmonella typhimurium LT2
tttacctcaagttaacttgaggaatt

SEQ ID NO: 152 - DNA, 26 bp, Salmonella enterica subsp. enterica serovar Typhi str. CT18
tttacctcaagttaacttgaggaatt

SEQ ID NO: 153 - DNA, 26 bp, Salmonella enterica subsp. enterica serovar Typhi Ty2
tttacctcaagttaacttgaggaatt

SEQ ID NO: 154 - DNA, 26 bp, Shewanella amazonensis SB2B
ttgacctcaagttgacttgaagttgt

SEQ ID NO: 155 - DNA, 26 bp, Shewanella denitrificans OS217
ttgacctcaagttagcttgagctttt

SEQ ID NO: 156 - DNA, 26 bp, Shewanella denitrificans OS217
tttacctcaagctaacttgaggtttt

SEQ ID NO: 157 - DNA, 26 bp, Shewanella frigidimarina NCIMB 400
tttacctcaagttagctttaggtatt

SEQ ID NO: 158 - DNA, 26 bp, Shewanella loihica PV-4
ttgacctcaagtcgacttgaagttgt

SEQ ID NO: 159 - DNA, 26 bp, Shigella dysenteriae Sd197
tttacctcaagttaacttgaggaatt

SEQ ID NO: 160 - DNA, 26 bp, Shigella flexneri 2a str. 2457T
tttacctcaagttaacttgaggaatt

SEQ ID NO: 161 - DNA, 26 bp, Shigella flexneri 2a str. 301
tttacctcaagttaacttgaggaatt

SEQ ID NO: 162 - DNA, 26 bp, Shigella flexneri 5 str. 8401
tttacctcaagttaacttgaggaatt

SEQ ID NO: 163 - DNA, 26 bp, Shigella sonnei Ss046
tttacctcaagttaacttgaggaatt

SEQ ID NO: 164 - DNA, 26 bp, Silicibacter pomeroyi DSS-3
ttgacctaaagttaggtttagaaatt

SEQ ID NO: 165 - DNA, 26 bp, Silicibacter sp. TM1040 mega plasmid
ttgagctaaagttaggtttagaaatt

SEQ ID NO: 166 - DNA, 26 bp, Silicibacter sp. TM1040
ttgatctaaagtgagcttgaggaatt

SEQ ID NO: 167 - DNA, 26 bp, Sinorhizobium medicae WSM419
ttgacctcaactttagttgaggttct

SEQ ID NO: 168 - DNA, 26 bp, Sinorhizobium meliloti 1021
ttgacctcaactatagttgaggttct

SEQ ID NO: 169 - DNA, 26 bp, Sinorhizobium meliloti 1021
ttgacctcaagtacggttgaggtcgc

SEQ ID NO: 170 - DNA, 26 bp, Sorangium cellulosum ′So ce 56′
ttgacctcaagttgacttgagcttgc

SEQ ID NO: 171 - DNA, 26 bp, Sphingopyxis alaskensis RB2256
ttgacctccactgcacttgagctttc

SEQ ID NO: 172 - DNA, 26 bp, Streptomyces avermitilis MA-4680
ttgacctgaagtttggttgaggttgc

SEQ ID NO: 173 - DNA, 26 bp, Streptomyces avermitilis MA-4680
ttgacctcaagattgcttgaggttct

SEQ ID NO: 174 - DNA, 26 bp, Streptomyces avermitilis MA-4680
ttgacctcaagattggttgaggtacc

SEQ ID NO: 175 - DNA, 26 bp, Streptomyces coelicolor A3(2)
ttgacctcaagcaaacttgaggtacc

SEQ ID NO: 176 - DNA, 26 bp, Streptomyces coelicolor A3(2)
ttgacctcaagcaggcttgaggtcgt

SEQ ID NO: 177 - DNA, 26 bp, Thermobifida fusca YX
ttgacctcaactttggttgaggtttt

SEQ ID NO: 178 - DNA, 26 bp, Thermobifida fusca YX
ttgacctcaacctaacctgagatttg

SEQ ID NO: 179 - DNA, 26 bp, Trichodesmium erythraeum IMS101
ttaacctcaagttaactttagatagt

SEQ ID NO: 180 - DNA, 26 bp, Vibrio cholerae O1 biovar eltor str. N16961 chromosome II
tttacctaaagttaacttgaggtatt

SEQ ID NO: 181 - DNA, 26 bp, Vibrio cholerae O395 chromosome 1
tttacctaaagttaacttgaggtatt

SEQ ID NO: 182 - DNA, 26 bp, Vibrio harveyi ATCC BAA-1116
ttgacctaaagttaacttgagctttt

SEQ ID NO: 183 - DNA, 26 bp, Vibrio harveyi ATCC BAA-1116
ttgacctcaacttcggttgaggtttt

SEQ ID NO: 184 - DNA, 26 bp, Vibrio harveyi ATCC BAA-1116
tttaccttaacttaacttgaggtttt

SEQ ID NO: 185 - DNA, 26 bp, Vibrio harveyi ATCC BAA-1116
ttaacctcaacctagcttgaggtttt

SEQ ID NO: 186 - DNA, 26 bp, Vibrio parahaemolyticus RIMD 2210633 chromosome II
ttgacctaaaccaaacttgaggtttt

SEQ ID NO: 187 - DNA, 26 bp, Vibrio parahaemolyticus RIMD 2210633 chromosome II
tttacctcaagtttacttgaggttct

SEQ ID NO: 188 - DNA, 26 bp, Vibrio parahaemolyticus RIMD 2210633 chromosome II
tttacctaaacttagcttgaggttct

SEQ ID NO: 189 - DNA, 26 bp, Vibrio parahaemolyticus RIMD 2210633 chromosome II
tttacctcgatttaacttgaggtttt

SEQ ID NO: 190 - DNA, 26 bp, Vibrio vulnificus CMCP6 chromosome II
ttgacctcaagttaacttgaggaatt

SEQ ID NO: 191 - DNA, 26 bp, Vibrio vulnificus CMCP6 chromosome II
tttacctcaagttaagttgagctttt

SEQ ID NO: 192 - DNA, 26 bp, Vibrio vulnificus CMCP6 chromosome II
tttacctcaatttaacttgaggtttt

SEQ ID NO: 193 - DNA, 26 bp, Vibrio vulnificus YJ016 chromosome II
ttgacctcaagttaacttgaggaatt

SEQ ID NO: 194 - DNA, 26 bp, Vibrio vulnificus YJ016 chromosome II
tttacctcaagttaagttgagctttt

SEQ ID NO: 195 - DNA, 26 bp, Vibrio vulnificus YJ016 chromosome II
tttacctcaatttaacttgaggtttt

SEQ ID NO: 196 - DNA, 26 bp, Xanthomonas campestris pv. campestris str. 8004
ttgacctcaaccttggttgaggcagg

SEQ ID NO: 197 - DNA, 26 bp, Xanthomonas campestris pv. campestris str. ATCC 33913
ttgacctcaaccttggttgaggcagg

SEQ ID NO: 198 - DNA, 26 bp, Xanthomonas campestris pv. vesicatoria str. 85-10
ttgacctcaacttaggttgaggcagg

SEQ ID NO: 199 - DNA, 26 bp, Xanthomonas axonopodis pv. citri str. 306
ttgacctcaacttaggttgaggcagg

SEQ ID NO: 200 - DNA, 26 bp, Xanthomonas axonopodis pv. citri str. 306
ttgacctcaactgcgcttgaggtcgt

SEQ ID NO: 201 - DNA, 26 bp, Xanthomonas oryzae pv. oryzae MAFF 311018
ttgacctcaactcaggttaaggcagg

SEQ ID NO: 202 - DNA, 26 bp, Zymomonas mobilis subsp. mobilis ZM4
ttgacctcaagttaacttgaggttca

SEQ ID NO: 203 - DNA, 74 bp, Escherichia coli K12
  misc_feature (38)..(71): n is a, c, g, or t
tttacctcaagttaacttgaggaattatactccccaannnnnnnnnnnnnnnnnnnnnnnnnnnnnnnnnnatg

SEQ ID NO: 204 - DNA, 18 bp, Escherichia coli K12
cctcaagttaacttgagg

SEQ ID NO: 205 - DNA, 26 bp, Escherichia coli K12
tttacctcaagttaacttgaggaatt

SEQ ID NO: 206 - DNA, 26 bp, Pseudomonas aeruginosa
tttacctcaagttaacttgagctatc

SEQ ID NO: 207 - DNA, 26 bp, Pseudomonas aeruginosa
ttgacctcaacttaacttgaggtttt

SEQ ID NO: 208 - DNA, 26 bp, Pseudomonas aeruginosa
ttgacctcaagtttgcttgaggtttt

SEQ ID NO: 209 - DNA, 26 bp, Chromobacterium violaceum
ttgacttcaagttaacttgaactttg

SEQ ID NO: 210 - DNA, 26 bp, Hyphomonas neptunium
ttgatctaaagtgaacttgagattgt

SEQ ID NO: 211 - DNA, 26 bp, Saccharopolyspara erythraea
ttgacctccagccaactcgaagtttc

SEQ ID NO: 212 - DNA, 26 bp, Xanthamonas axonapodis
ttgacctcaacttaggttgaggcagg

SEQ ID NO: 213 - DNA, 26 bp, Silicibacter pomeroyl DSS-3
ttgacctaaagttaggtttagaaatt

SEQ ID NO: 214 - DNA, 26 bp, Maricaulls maris MC510
ttgacctcaaccaaggttaagcaatt

SEQ ID NO: 215 - DNA, 26 bp, Mycobacterium smegmatis
ttcacctgaagtaaggtttaggtgtc

SEQ ID NO: 216 - DNA, 26 bp, Mycobacterium vanbaalenii PYR-1
atgacctcaaccctagttgaggtgac

SEQ ID NO: 217 - DNA, 26 bp, Consensus
  misc_feature (24)..(24): n is a, c, g, or t
ttgacctcaagttaacttgaggtntt

(19) United States
(12) Patent Application Publication
(10) Pub. No.: US 2010/0124554 A1
(43) Pub. Date: May 20, 2010

(54) REDOX-ACTIVE COMPOUNDS AND RELATED COMPOUNDS, COMPOSITIONS, METHODS AND SYSTEMS

(76) Inventors: Dianne K. Newman, Brookline, MA (US); Lars E.P. Dietrich, Cambridge, MA (US); Yun Wang, Evanston, IL (US)

Correspondence Address: Steinfl & Bruno, 301 N Lake Ave Ste 810, Pasadena, CA, 91101, US

(21) Appl. No.: 12/548,362

(22) Filed: Aug. 26, 2009

Related U.S. Application Data
(60) Provisional application No. 61/092,739, filed on Aug. 28, 2008.

(51) Int. Cl. A61K 39/40; A61K 31/7088; A61K 31/7036; A61K 38/08; A61K 31/7052; A61K 31/7048; A61K 38/12; A61K 31/7056; C12Q 1/02; A61P 31/04

(52) U.S. Cl. 424/170.1; 424/164.1; 514/44 A; 514/40; 514/41; 514/39; 514/37; 514/8; 514/28; 514/29; 514/11; 514/24; 435/29

(57) ABSTRACT

Compounds able to affect production and/or activity of a redox active compound, which include candidate therapeutic compounds and candidate compounds for enhancing power output of a microbial fuel cell, and related compositions, methods and systems.

### RELATED APPLICATIONS

The present application claims priority of U.S. provisional Application No. 61/092,739 entitled “Inhibitors of Redox-Active Compounds and Uses Therefor” filed on Aug. 28, 2008 with Docket No. M0656.70167US00 incorporated herein by reference in its entirety.

### FIELD

The present disclosure relates to redox active compounds and related compounds compositions methods and systems.

### BACKGROUND

Redox active compounds such as redox-active pigments are among compounds produced by certain organisms and in particular unicellular organisms, such as bacteria. In particular, it is thought that bacteria excrete redox-active pigments as antibiotics to inhibit competitors. Several activities are associated to redox-active compounds in bacteria. For example, in Pseudomonas aeruginosa, the endogenous antibiotic pyocyanin activates SoxR, a transcription factor conserved in Proteo- and Actinobacteria. In Escherichia coli, SoxR regulates the superoxide stress response.

### SUMMARY

Provided herein are compounds, composition methods and systems based at least in part on the discovery that redox active molecules affect the morphology of colonies of redox-active organisms such as unicellular organisms (e.g., bacteria). Provided herein are also compounds, composition methods and systems based at least in part on the discovery that redox-active molecules help redox-active organisms survive under anaerobic conditions, for example, in the context of an infection.

In some aspects, the disclosure relates to methods and systems for identifying a compound able to affect production and/or activity of redox active compounds. The methods comprise (i) contacting a test colony of redox active unicellular organism with a test agent; (ii) after contacting, determining a morphological parameter of the test colony; and (iii) comparing the morphological parameter to a reference, wherein the comparison is indicative of whether or not the test agent is a compound able to affect the production and/or activity of a redox active compound in the unicellular organism. The systems comprise at least two of a test agent, a redox-active bacterial colony, a control bacterial colony, and suitable reagents and means to perform the related methods herein described.

In some aspects, the disclosure relates to methods and systems for identifying a candidate therapeutic compound. The methods involve (i) contacting a test colony of redox active bacteria with a test agent; (ii) after contacting, determining a morphological parameter of the test colony; and (iii) comparing the morphological parameter to a reference, wherein the comparison is indicative of whether or not the test agent is a candidate therapeutic compound. The systems comprise at least two of a test agent, a redox-active bacterial colony, a control bacterial colony, and suitable reagents and means to perform the related methods herein described.

In some aspects, the disclosure relates to methods of treating a bacterial infection in a patient. The methods comprise administering to the patient an inhibitor of redox active compounds in an effective amount.

In some aspects, the disclosure provides methods of treating a bacterial infection in a patient. The methods comprise administering to the patient an effective amount of an inhibitor of redox active compounds in combination with an antibiotic compound.

In some aspects, the disclosure relates to systems of treating a bacterial infection in a patient. The systems comprise an inhibitor of a redox-active compound and an antibiotic compound as a combined preparation for the simultaneous, separate or sequential administration in a therapeutic or prophylactic treatment of a bacterial infection.

In some aspects, the disclosure relates to compositions, and in particular, pharmaceutical composition, comprising an inhibitor of a redox-active compound and a suitable carrier. In embodiments wherein the compositions are pharmaceutical compositions the suitable carrier is a pharmaceutically acceptable carrier.

In some aspects, the disclosure relates to methods and systems for identifying candidate bacteria for enhancing power output of a microbial fuel cell. The methods involve (i) mutagenizing a test bacterial colony; (ii) determining morphology of the test bacterial colony; and (iii) comparing the morphology of the test bacterial colony to a reference, wherein the comparison is indicative of whether or not the test bacterial colony is comprised of a candidate bacterium for enhancing power output of a microbial fuel cell. The systems comprise at least two of a test agent, a redox-active bacterial colony, a control bacterial colony, and suitable reagents and means to perform the related methods herein described.

In some aspects, the disclosure relates to methods for identifying a candidate compound for enhancing power output of a microbial fuel cell. The methods involve (i) contacting a redox active bacterial colony with a test agent; (ii) determining morphology of the redox active bacterial colony; and (iii) comparing the morphology of the redox active bacterial colony to a reference, wherein the comparison is indicative of whether or not the test agent is a candidate compound for enhancing power output of a microbial fuel cell. The systems comprise at least two of a redox-active bacterial colony, a control bacterial colony, and suitable reagents and means to perform the related methods herein described.

Compounds, compositions, methods and systems herein described that reduce the production and/or activity of redox-active molecules by an organism, can be useful, in some embodiments, to prevent or treat an infection by that organism.

Compounds, compositions, methods and systems herein described that enhance the production and/or activity of redox active molecules by an organism can be useful in some embodiments in connection with use of microbial fuel cells.

Compounds, compositions, methods and systems herein described that enhance the production and/or activity of redox active molecules by an organism can be useful in some embodiments in connection with use in corrosion control.

### BRIEF DESCRIPTION OF DRAWINGS

The accompanying drawings, which are incorporated into and constitute a part of this specification, illustrate one or more embodiments of the present disclosure and, together with the detailed description, the examples and the Appendix A, serve to explain the principles and implementations of the disclosure.

FIG. 1 shows a schematic representation of the organization of Sox regulons and the SoxRbox consensus sequence in redox-active organism according to some embodiments herein described. In particular, in the illustration of FIG. 1A the P. aeruginosa SoxR regulon differs from the E. coli paradigm. In E. coli, the SoxR homodimer binds to the soxRbox in the soxS promoter region (see also FIG. 1B). soxR and soxS are divergently transcribed. The binding of reduced SoxR to the soxRbox represses expression of soxR and soxS. Oxidation of the SoxR [2Fe-2S] cluster induces a conformational change that allows transcription of soxS (E. Hidalgo, V. Leautaud, B. Demple, The EMBO Journal 17,2629 (May 1, 1998)). SoxS regulates genes involved in superoxide tolerance and detoxification. In contrast, in P. aeruginosa, the gene adjacent to soxR encodes a putative monooxygenase. Two additional soxRboxes, found elsewhere in the P. aeruginosa genome, regulate expression of putative drug transporters. In the illustration of FIG. 1B SoxRbox consensus sequence. SoxR binding sites (soxRboxes) are palindromic sequences positioned between the −35 and −10 promoter elements. The promoter region upstream of E. coli's soxS is shown. +1 marks the transcriptional start site, ATG the start codon in soxS. A position weight matrix was generated based on 12 soxRbox sites from diverse bacteria. A modified version of the program pyscangenes.py (C. T. Brown and C. G. Callan, Jr., Proc Natl Acad Sci USA 101 (8), 2404 (2004)) was used to find the site energy of all motifs in 616 genomes (Table 1). Dots indicate nucleotides that are identical to those in the E. coli soxRbox.

FIG. 2 shows a schematic representation of the distribution between SoxR and SoxS and the gene categories regulated by SoxR in redox active organisms according to some embodiments herein described. In particular, FIG. 2A schematically shows the distribution of SoxR and SoxS among phyla of the domain Bacteria. A BLAST search for E. coli SoxR and SoxS was performed and SoxS was found only in enterics. SoxR homologs were identified in 165 alpha-, beta-, delta-, and gamma-Proteobacteria and Actinobacteria. All of these homologs contain the SoxR-specific cysteine motif CI[G/Q]CGC[L/M][S/L]xxxC required for binding of the [2Fe-2S] cluster. The number of hits within respective phyla is indicated, followed by the total number of genomes surveyed. Members of these phyla (in black) are noted for their ability to produce and excrete redox-active small molecules, such as phenazines (J. M. Turner, A. J. Messenger, Adv Microb Physiol 27, 211 (1986)) and actinorhodin (K. F. Chater, Philos Trans R Soc Lond B Biol Sci 361, 761 (2006)). Representative structures are shown. The tree was constructed using the ARB neighbor joining method from 16S rRNAs of 604 bacterial species. The bar represents 0.1 base substitutions per nucleotide. FIG. 2B shows diagram illustrating gene categories regulated by SoxR. Only in enterics are soxRboxes located upstream of soxS, confirming the uniqueness of this network. In all other soxR-containing Proteo- and Actinobacteria soxRboxes are mainly found upstream of five gene types as indicated. 100% correspond to 16 alpha-Proetobacteria, 18 beta-Proteobacteria, 27 enteric, 38 non enteric gamma-Proteobacteria or 22 Actinobacteria. “Dehydr.” stands for putative dehydrogenases, “oxygen.” putative mono- or di-oxygenases, “L-PSP” putative L-PSP ribonucleases, and “methyl./acetylase” putative methyl- or acetyl-transferases. Additional annotation information can be found at http://soxRbox.mit.ed

FIG. 3 shows a schematic illustration of a phylogenetic tree based on SoxR orthologs in redox-active organisms according to some embodiments herein described. Applicants constructed a phylogenetic tree based on 123 SoxR orthologs using the desktop software CLC protein workbench 3. The sequences were aligned with the ClustalW algorithm (gap open penalty=35; gap extension penalty=0.75; Scoring matrix=GONNET). Using this alignment we created a UPGMA (algorithm which assumes a constant rate of evolution) tree and performed a bootstrap analysis with 1000 replicates.

FIG. 4 shows redox-active molecules produced by redox-active organisms according to some embodiments herein described. In particular FIG. 4A shows structures of undecylprodigiosin and actinorhodin. Both compounds are produced by the S. coelicolor A3(2) parent strain M145, but not by S. coelicolor A3(2) M512 (ΔredD ΔactII-ORF4 (B. Floriano, M. Bibb, Mot Microbiol 21, 385 (July, 1996)). FIG. 4B shows a picture illustrating spore suspensions of the S. coelicolor A3(2) parent strain M145 and M512 were distributed over cellophane sheets that were placed on R5⁻ plates and incubated at 30° C. After three days the M145 spread turned red due to the production of undecylprodigiosin, after four days it turned blue due to actinorhodin production. At day 5 we lifted the cellophane sheets of the agar plates, scraped off and prepared RNA for quantitative RT-PCR (FIG. 2).

FIG. 5 shows a schematic illustration of putative SoxR regulon in a redox-active organism according to some embodiments herein described. In particular, in Panel A, the putative S. coelicolor A3(2) SoxR regulon specifically upregulated by pigments is schematically shown. Genes predicted to be regulated by SoxR are shown in grey. Panel B shows a chart illustrating RNA extracted from plate-grown S. coelicolor A3(2) M145 and the pigment-null mutant M512 was used to generate cDNA for quantitative RT-PCR (for more information see SOM). Signals were standardized to SCO4548 (30). The experiment was done in triplicate, and data reported represent the mean±the standard deviation. SoxR itself (SCO1697) was also tested for changes in gene expression.

FIG. 6 effects of a redox-active molecule on the morphology of a redox active organism according to some embodiments herein described. Phenazine production modulates colony morphology in P. aeruginosa PA14. P. aeruginosa cultures were spotted onto agar plates containing Congo Red and Coomassie Blue, and incubated at 20° C. for six days. The phenazine null strain (Δphz) started to wrinkle on day 2, the wildtype (wt) wrinkled on day 3, and the soxR and mexGHI-opmD deletion strains wrinkled on day 5, while a pyocyanin-over producer (DKN370) remained smooth and white after 6 days.

FIG. 7 shows effects of a redox-active molecule on the morphology of a redox active organism according to some embodiments herein described. In particular, Panel A shows a chart illustrating surface coverage of 35 colonies per strain monitored over 8 days. Error bars represent the standard deviation. Panel B shows a chart illustrating the concentration of pyocyanin release from three colonies into 10 ml agar supplemented with Congo Red and Coomassie Blue. After 5 days of growth at room temperature the cells were scraped off, pyocyanin was extracted from the agar using chloroform, and extracts were analyzed by HPLC. The data reported represent the mean±the standard deviation. Panel C shows spore suspensions of S. coelicolor A3(2) M145 and the pigment mutant M512 were spotted and incubated for five days on R5⁻ medium at room temperature. The pigment mutant exhibits a wrinkled morphology, whereas the wild type takes on a smoother phenotype. Bar is 0.5 cm.

FIG. 8 shows effects of a redox-active molecule on the morphology of a redox active organism according to some embodiments herein described. In particular, in FIG. 8 is shown how the phenazine null mutant forms smooth colonies in the presence of exogenous phenazines. Panel A, shows PA 14 wt and the phenazine null mutant were spotted onto 1% tryptone, 1% agar plates that were supplemented with 50 μM HPLC-purified pyocyanin or an equal volume of water. Panel B shows 50 μl cell suspensions of PA14 phenazine null mutant and the phenazine overproducer were streaked onto 1% tryptone, 1% agar plates (supplemented with Congo Red and Coomassie Blue). Image was taken after 5 days.

FIG. 9 shows effects of a redox-active molecule on the morphology of a redox active organism according to some embodiments herein described. In particular, Panel A shows a schematic of the genomic localization of mexGHI-opmD, phzM, phzA1-G1, and phzS in P. aeruginosa PA14. Panel B shows deletion of the RND efflux pump mexGHI-opmD results in an extended lag phase. This phenotype is dependent on phenazine production as a mexGHI-opmD deletion in the phenazine null-mutant does not show this phenotype. All strains were inoculated from late stationary phase cultures into 5 ml LB to an OD (600 nm) of 0.05, then incubated shaking (225 rpm) at 37° C.

FIG. 10 shows charts illustrating effects of a redox-active molecule on survival of a redox active organism according to some embodiments herein described. Redox active molecule PYO (Panel A), PCA (Panel B), and 1-OHPHZ (Panel C) functioned as an electron shuttle (▪) to promote anaerobic survival of P. aeruginosa PA14 Δphz mutant, when cells were incubated anaerobically in the MOPS media containing 20 mM D-glucose, ˜90 μM phenazine (PYO, or PCA, or 1-OHPHZ), and with the graphite rod working electrode poised at +0.2 V vs. NHE. Survival was determined as viable cell counts as measured by colony forming units (CFU) on LB agar plates. CFU of Δphz anaerobic incubations without phenazine (∇), or poised potential (Δ), or both (⋄) served as controls. Error bars represent standard deviations from at least triplicate samples in each experimental set. Plots represent results from at least three independent experiments.

FIG. 11 shows a chart illustrating effects of a redox-active molecule on survival of a redox active organism according to some embodiments herein described. Anaerobic survival of P. aeruginosa PA14 Δphz mutant without D-glucose ( ) and in the presence of 20 mM D-glucose (▪), for cells incubated in the MOPS media containing ˜90 μM PYO, and with the graphite rod working electrode poised at +0.2 V vs. NHE. Survival was determined as viable cell counts as measured by colony forming units (CFU) on LB agar plates. Error bars represent standard deviations from triplicate samples in each experimental set. Plots represent results from two independent experiments.

FIG. 12 shows a chart illustrating effects of a redox-active molecule on survival of a redox active organism according to some embodiments herein described. Cyclic voltammetry (CV) of P. aeruginosa PA14 Δphz mutant cultures incubated anaerobically in 100 mL MOPS media containing 20 mM glucose, supplemented with 90 μM PYO (dark trace) versus no PYO (light trace), displayed PYO as the only electrochemically active component with single anodic (oxidation) and cathodic (reduction) peaks characteristic of itself. CV experiments were performed at 100 mV/s of electrodes consisting of a stationary gold disk working electrode, a Ag/AgCl reference electrode, and a Pt counter electrode

### DETAILED DESCRIPTION

Certain aspects of the disclosure relate, at least in part, to the discovery that redox-active molecules affect the morphology of colonies of unicellular organisms (e.g., bacteria). Accordingly, in some embodiments the morphology of a colony (e.g., a bacterial colony) may be used as a marker to evaluate the effect of a compound (e.g., a test compound) on the production (e.g., synthesis, secretion, and/or transformation) and/or activity of redox-active molecules in an organism or a colony of organisms.

In particular, aspects of the present disclosure relate to screens for identifying compounds or candidate compounds that can affect production and/or activity of a redox-active compound in an organism and in particular may be useful to inhibit the production of one or more redox-active molecules in the organism. A redox-active molecule can be identified as a molecule that can be oxidized and reduced (e.g., using an electrode scan). In some embodiments, a redox-active molecule may be a redox-active pigment, a phenazine, or other redox-active molecules as described in more detail herein as well as additional molecules identifiable by a skilled person.

In some embodiments, a screen may involve assaying the effect of a test compound on the morphology of a colony of organisms (e.g., a bacterial colony). Various morphological parameters can be considered in determining whether a compound is able to affect production and/or activity of a redox-active molecule in an organism. For example, in some embodiments, a compound that increases the surface area and/or rugosity of a colony (e.g., a bacterial colony) may be used to identify compounds that inhibit the production of a redox-active molecule.

In some embodiments, a screen may involve assaying the effect of a test compound on the color of one or more organisms that expresses a redox-active pigment (e.g., when grown on a solid support or in a liquid culture). In some embodiments, a color assay may be used to identify a compound that inhibits production of a phenazine (e.g., by screening for a compound that causes a lack of color, for example a lack of blue color). In some embodiments, a color assay may be used to identify a compound that inhibits the reduction of a redox-active pigment (e.g., by screening for a slower conversion of one color to another or of a color to a lack of color, for example a slower conversion of blue to clear). In some embodiments, a color assay may be used to identify a compound that slows the transport of a redox-active pigment (e.g., by screening for a lower intensity of a color, for example a lower intensity of blue).

In some embodiments, the screen is directed to identify a compound that is able to inhibit production and/or activity of redox molecules in a redox active organism (herein also inhibitor). Exemplary compounds that inhibit production of a redox-active molecule include but are not limited to compounds that are able to prevent or reduce biosynthesis, transport and/or chemical modification of a redox active molecule in a redox-active organism of interest. Exemplary compounds that inhibit the activity of redox molecules include but are not limited to compounds that are able to prevent or reduce the biological activity of a redox active molecule in the redox-active organism of interest. In several embodiments, the biosynthesis, transport, chemical modification and/or biological activity used as a reference to detect prevention or reduction are the ones associated to a viable status of the redox-active organism. Techniques suitable to detect inhibition of production and/or activity of redox molecules in a redox active organism are identifiable by a skilled person upon reading of the present disclosure and will not be further described in detail.

Exemplary inhibitors include but are not limited to compounds that are able to convert a redox-active molecule from a biologically active form to a biologically inactive form or to convert the redox-active molecule from a biologically active form to a biologically less or reduced active form, and compounds whose interaction with the redox-active organism results in a total or partial deletion of the biologically active molecule. For example, inhibition of a redox-active molecule can be performed by deleting or mutating a native or heterologous polynucleotide encoding for the a redox-active molecule in the redox-active organism, by deleting or mutating a native or heterologous polynucleotide encoding for an enzyme involved in the pathway for the synthesis of the a redox-active molecule in the redox-active organism, by activating a further a native or heterologous molecule that inhibits the expression of the a redox-active molecule in the redox-active organism.

Some of the embodiments, where the screen is directed to identify an inhibitor of a redox molecule in a redox active organism relate, at least in part, to the discovery that redox-active molecules help organisms survive under anaerobic conditions, for example, in the context of an infection. Accordingly in some embodiments, candidate compounds that are able to inhibit the production and/or activity of a redox-active molecule in an organism are identified and used as a candidate therapeutic compound.

Additionally, in some embodiments, a compound can also be screened for its ability to reduce the viability of a redox-active organism under anaerobic conditions. In particular, in certain embodiments, a candidate compound that is first identified as an inhibitor of production and/or activity of redox-active molecules in an organism using a morphological and/or color screen described herein may be subsequently screened or evaluated to determine whether or confirm that the candidate compound reduces viability of the organism under anaerobic conditions.

In some embodiments, the screen is directed to identify a compound that is able to enhance production and/or activity of redox-active molecules in a redox-active organism (herein also enhancer). Compounds that enhance production of a redox-active molecule include but are not limited to compounds that are able to increase biosynthesis, transport and/or chemical modification of a redox active molecule in a redox-active organism of interest. Compounds that enhance the activity of redox molecules include but are not limited to compounds that are able to increase the biological activity of a redox active molecule in the redox-active organism of interest. In several embodiments, the biosynthesis, transport, chemical modification and/or biological activity used as a reference to detect increase are the ones associated to a viable status of the redox-active organism. Techniques suitable to detect enhancement of production and/or activity of redox molecules in a redox active organism are identifiable by a skilled person upon reading of the present disclosure and will not be further described in detail.

In other embodiments, the methods herein described can be used to identify organism, e.g. mutagens, that have an enhanced production and/or activity of a redox-active molecules. Exemplary organisms having an enhanced production and/or activity of redox-active molecules as used herein with reference to a redox-active molecule, indicates redox-active organism having any modification in the genome and/or proteome that increases the biological activity of the redox-active molecule in the redox-active organism. Exemplary enhancements of redox-active molecule include but are not limited to modifications that results in the conversion of the redox-active molecule from a biologically inactive form to a biologically active form and from a biologically active form to a biologically more active form, and modifications that result in the expression of the redox-active active molecule in a redox-active organism wherein the biologically active molecule was previously not expressed. For example, enhancement of a redox-active active molecule can be performed by expressing a native or heterologous polynucleotide encoding for the biologically active molecule in the organism, by expressing a native or heterologous polynucleotide encoding for an enzyme involved in the pathway for the synthesis of the redox-active molecule in the organism, by expressing a native or heterologous molecule that enhances the expression of the redox-active molecule in the microorganism.

Organisms and compounds that are identified in outcome of these methods can be used in connection with applications where enhanced production and/or activity of redox-active molecules by redox active organisms is desired, such as microbial fuel cells where the compound and/or organism can be used for enhancing power output of a microbial fuel cell. Some of those embodiments relate, at least in part, to the discovery that redox-active molecules help organisms survive under anaerobic conditions, for example in the context of microbial fuel cells or applications related to beneficial biofilms used in corrosion control.

In several embodiments of methods herein described, wherein a compound is screened, screening of the compound can be performed by (i) contacting a test colony of a redox active unicellular organism, and in particular a bacteria, with a test agent and (ii) after contacting determining the morphological parameter of the test colony.

In several embodiments, wherein an organism, such as bacteria, is screened, screening of candidate bacteria can be performed by (i) mutagenizing a test bacterial colony; and (ii) after mutagenizing determining morphology of the test bacterial colony.

In some embodiments, the methods herein described involve growing the unicellular organism, in particular bacteria, on an agar plate, before after and/or between steps (i) and (ii).

In some embodiments of the methods herein described where a compound is screened and the redox-active organism is bacteria, contacting of a test agent is performed when the bacteria are in late exponential-phase growth and in particular in late exponential phase planktonic growth.

In some embodiments of the methods herein described, the morphological parameter is selected from: texture (smooth/wrinkled), size (spread/compact), area, perimeter, perimeter to area ratio, volume, major axis length, minor axis length, equivalent ellipse area, area to equivalent ellipse area ratio, bounding box length, bounding box width, bounding box length to width ratio, bounding box area, convex hull area, convex hull perimeter, average radius, average fiber length, average fiber width, volume, and rugosity.

In some embodiments of the methods herein described, steps (i) and (ii) are repeated at two or more intervals. In certain embodiments, the two or more intervals are selected from: about 0, about 1, about 2, about 3, about 4, about 5, about 6, about 7, about 8, about 9, or about 10 days after step (i).

In some embodiments of methods herein described, where the unicellular organism is a bacterium, the redox active bacterium is an Actinobacterium or a Proteobacterium. In certain embodiments, the redox active bacterium is a Pseudomonas species, optionally wherein the Pseudomonas species is selected from P. aureofaciens, P. fluorescens, and P. chlororaphis.

In several embodiments, the methods further comprise (iii) comparing the morphological parameter determined in outcome of (ii) to a reference. In particular the reference is associated with a production and/or activity level of redox-active compounds in the organism that is selected as a basis to evaluate the ability to modify said production and/or activity by the candidate compound, according to the experimental design. Additionally in certain embodiments the reference is associated with a viability or metabolic status of the redox active organism.

In some embodiments, the reference is the value of the morphological parameter in a control colony.

In certain embodiments of methods herein described, the control colony is a colony of redox active bacteria that has not been contacted with the test agent. In certain embodiments of the methods herein described, the control colony is a colony of redox active bacteria that has been contacted with an inactive control molecule.

In certain embodiments, wherein screening of a compound is performed to identify an inhibitor of redox active molecules and in particular a candidate therapeutic compound, the control colony is a colony of redox active bacteria having a mutation in the SoxR locus, optionally wherein the mutation is a deletion in, or of, the SoxR locus. In certain embodiments, wherein screening of a compound is performed to identify an inhibitor of redox active molecules and in particular a candidate therapeutic compound, the control colony is a colony of redox active bacteria having a mutation in the mexGHI-opmD operon, optionally wherein the mutation is a deletion in, or of, the mexGHI-opmD operon.

In some embodiments, wherein screening of a compound is performed to identify an inhibitor of redox active molecules and in particular a candidate therapeutic compound, a statistically significant difference between the morphological parameter and the reference indicates that the test agent is a candidate therapeutic compound. In particular, in some embodiments, the morphological parameter is area, and a statistically significant increase in area of the test colony compared with the control colony indicates that the test agent is a candidate therapeutic compound. In one embodiment, the morphological parameter is shape, and a statistically significant increase in spread of the shape of the test colony compared with the control colony indicates that the test agent is a candidate therapeutic compound. In one embodiment, the morphological parameter is texture, and a statistically significant increase in the wrinkledness of the test colony compared with the texture of the control colony indicates that the test agent is a candidate therapeutic compound.

In some embodiments of the methods herein described, wherein screening is performed to identify an inhibitor of redox active molecules and in particular to identify a candidate therapeutic compound, the control colony is a colony of redox active bacteria having a mutation in a phenazine biosynthetic gene, optionally wherein the mutation is a deletion in, or of, a phenazine biosynthetic gene. In certain embodiments, the phenazine biosynthetic gene is phzM, phzA1-G1 or phzS. In some embodiments of the methods herein described, wherein screening of a compound is performed to identify an inhibitor of redox active molecules and in particular a candidate therapeutic compound, the control colony is a colony of redox active bacteria having a mutation in a gene that controls the chemical transformation affinities. In some embodiment, including embodiments wherein the control colony has a mutation in a phenazine biosynthetic gene, a lack of a statistically significant difference between the morphological parameter and the reference indicates that the test agent is a candidate therapeutic compound.

In some embodiments of the methods herein described, wherein screening is performed to identify a compound for and/or an organism having, an enhanced production and/or activity of redox active molecules, the control colony is a colony of redox active bacteria that has been contacted with an exogenous phenazine compound. In certain embodiments, wherein screening is performed to identify a compound for and/or an organism having, an enhanced of production and/or activity of redox active molecules, the control colony is a colony of redox active bacteria having a mutation in the SoxR locus, optionally wherein the mutation is a deletion in, or of, the SoxR locus. In certain embodiments, wherein screening is performed to identify a compound for and/or an organism having, an enhanced of production and/or activity of redox active molecules, the control colony is a colony of redox active bacteria having a mutation in the mexGHI-opmD operon, optionally wherein the mutation is a deletion in, or of, the mexGHI-opmD operon. In certain embodiments, a lack of a statistically significant difference between the morphological parameter and the reference indicates that the test agent is a candidate compound for enhancing power output of a microbial fuel cell.

In some embodiments of methods herein described, wherein screening is performed to identify a compound for and/or an organism having, an enhanced production and/or activity of redox active molecules, the control colony is a colony of redox active bacteria having a mutation in a phenazine biosynthetic gene, optionally wherein the mutation is a deletion in, or of, a phenazine biosynthetic gene. In certain embodiments, the phenazine biosynthetic gene is phzM, phzA1-G1 or phzS. In certain embodiments, wherein screening is performed to identify a compound for and/or an organism having, an enhanced production and/or activity of redox active molecules, the morphological parameter is shape. In one embodiment, a statistically significant increase in compactness of the shape of the test colony compared with the control colony indicates that the test agent is a candidate compound for enhancing power output of a microbial fuel cell. In certain embodiments, wherein screening is performed to identify a compound for and/or an organism having, an enhanced production and/or activity of redox active molecules, the morphological parameter is texture. In one embodiment, wherein screening is performed to identify a compound for and/or an organism having, an enhanced production and/or activity of redox active molecules, a statistically significant increase in the smoothness of the texture of the test colony compared with the control colony indicates that the test agent is a candidate compound for enhancing power output of a microbial fuel cell.

In some embodiments of methods herein described, the reference is a predetermined value.

In certain embodiments, wherein screening is performed to identify an inhibitor of redox active molecules and in particular to identify a candidate therapeutic compound, the morphological parameter is maximum area, and wherein the predetermined value is about 2.5 cm². In one embodiment, if the maximum area is statistically significantly greater than the predetermined value of about 2.5 cm², then the test agent is identified as a candidate therapeutic compound. In one embodiment, if the maximum area is approximately equal to or greater than 3.0 cm², then the test agent is identified as a candidate therapeutic compound.

In some embodiments, wherein screening is performed to identify a compound for and/or an organism having, an enhanced production and/or activity of redox active molecules, the morphological parameter is maximum area, and the predetermined value of the maximum area is about 2.5 cm². In one embodiment, if the maximum area is statistically significantly less than the predetermined value of about 2.5 cm², then the test agent is identified as a candidate compound for enhancing power output of a microbial fuel cell. In one embodiment, if the maximum area is approximately equal to or less than 2 cm², then the test agent is identified as a candidate compound for enhancing power output of a microbial fuel cell.

In several embodiments, the methods can further comprise, determining based on a comparison result, whether or not the test agent is a compound able to affect the production and/or activity of a redox active compound in the unicellular organism, and in particular whether or not the test agent is an inhibitor of redox active molecules, whether or not the test agent is a candidate therapeutic compound, or whether or not the test agent is an enhancer of the production and/or activity of redox active molecules in redox active organism and in particular a candidate compound for enhancing power output of a microbial fuel cell.

In some embodiments, the methods described herein also involve acquiring a digital image of the test and/or control colony, optionally wherein the digital image is acquired through a microscope. In certain embodiments, the morphological parameter is determined from the digital image. In certain embodiments, the morphological parameter is selected from: texture (smooth/wrinkled), size (spread/compact), area, perimeter, perimeter to area ratio, rugosity, volume, major axis length, minor axis length, equivalent ellipse area, area to equivalent ellipse area ratio, bounding box length, bounding box width, bounding box length to width ratio, bounding box area, convex hull area, convex hull perimeter, average radius, average fiber length, and average fiber width.

The assay methods described herein are amenable to high-throughput screening (HTS) implementations. In some embodiments, the screening assays of the disclosure are high throughput or ultra high throughput (e.g., Fernandes, P. B., Curr Opin Chem Biol. 1998 2:597; Sundberg, S A, Curr Opin Biotechnol. 2000, 11:47). HTS refers to testing of up to, and including, 100,000 compounds per day. Whereas ultra high throughput (uHTS) refers to screening in excess of 100,000 compounds per day. The screening assays of the disclosure may be carried out in a multi-well format, for example, a 96-well, 384-well format, or 1,536-well format, and are suitable for automation. In the high throughput assays of the disclosure, it is possible to screen several thousand different compounds or compositions in a single day. In particular, each well of a microtiter plate can be used to run a separate assay against a selected test compound, or, if concentration or incubation time effects are to be observed, a plurality of wells can contain test samples of a single compound. It is possible to assay many plates per day; assay screens for up to about 6,000, 20,000, 50,000, or more than 100,000 different compounds are possible using the assays of the disclosure. Typically, HTS implementations of the assays disclosed herein involve the use of automation. In some embodiments, an integrated robot system consisting of one or more robots transports assay microplates between multiple assay stations for compound, cell and/or reagent addition, mixing, incubation, and finally readout or detection. In some aspects, an HTS system of the disclosure may prepare, incubate, and analyze many plates simultaneously, further speeding the data-collection process. High throughput screening implementations are well known in the art. Exemplary methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jörg Hüser, the contents of which are both incorporated herein by reference in their entirety. As described herein, compounds or compositions that substantially affect the growth, morphology and/or survival of a test cell can be discovered using the disclosed test methods.

The following provides examples of test compounds and is not meant to be limiting. Those of ordinary skill in the art will recognize that there are numerous additional types of suitable test compounds that may be tested using the methods, cells, and/or animal models of the disclosure. Test compounds can be small molecules (e.g., compounds that are members of a small molecule chemical library). The compounds can be small organic or inorganic molecules of molecular weight below about 3,000 Daltons. The small molecules can be, e.g., from at least about 100 Da to about 3,000 Da (e.g., between about 100 to about 3,000 Da, about 100 to about 2,500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The small molecules can be natural products, synthetic products, or members of a combinatorial chemistry library. A set of diverse molecules can be used to cover a variety of functions such as charge, aromaticity, hydrogen bonding, flexibility, size, length of side chain, hydrophobicity, and rigidity. Combinatorial techniques suitable for synthesizing small molecules are known in the art (e.g., as exemplified by Obrecht and Villalgrodo, Solid-Supported Combinatorial and Parallel Synthesis of Small-Molecular-Weight Compound Libraries, Pergamon-Elsevier Science Limited (1998)), and include those such as the “split and pool” or “parallel” synthesis techniques, solid-phase and solution-phase techniques, and encoding techniques (see, for example, Czarnik, A. W., Curr. Opin. Chem. Biol. (1997) 1:60). In addition, a number of small molecule libraries are publicly or commercially available (e.g., through Sigma-Aldrich, TimTec (Newark, Del.), Stanford School of Medicine High-Throughput Bioscience Center (HTBC), and ChemBridge Corporation (San Diego, Calif.).

Compound libraries screened using the new methods can comprise a variety of types of test compounds. A given library can comprise a set of structurally related or unrelated test compounds. In some embodiments, the test compounds are peptide or peptidomimetic molecules. In some embodiments, test compounds include, but are not limited to, peptide analogs including peptides comprising non-naturally occurring amino acids, phosphorous analogs of amino acids, amino acids having non-peptide linkages, or other small organic molecules. In some embodiments, the test compounds are peptidomimetics (e.g., peptoid oligomers, e.g., peptoid amide or ester analogues, D-peptides, L-peptides, oligourea or oligo-carbamate); peptides (e.g., tripeptides, tetrapeptides, pentapeptides, hexapeptides, heptapeptides, octapeptides, nonapeptides, decapeptides, or larger, e.g., 20-mers or more); cyclic peptides; other non-natural peptide-like structures; and inorganic molecules (e.g., heterocyclic ring molecules). Test compounds can also be nucleic acids.

The test compounds and libraries thereof can be obtained by systematically altering the structure of a first “hit” compound, also referred to as a lead compound, and correlating that structure to a resulting biological activity (e.g., a structure-activity relationship study).

Such libraries can be obtained using any of the numerous approaches in combinatorial library methods known in the art, including: peptoid libraries (libraries of molecules having the functionalities of peptides, but with a novel, non-peptide backbone which are resistant to enzymatic degradation but which nevertheless remain bioactive; see, e.g., Zuckermann, et al., J. Med. Chem., 37:2678-85 (1994)); spatially addressable parallel solid phase or solution phase libraries; synthetic library methods requiring deconvolution; the “one-bead on e-Compound” library method; and synthetic library methods using affinity chromatography selection (Lam, Anticancer Drug Des. 12:145 (1997)). Examples of methods for the synthesis of molecular libraries can be found in the art, for example in: DeWitt et al., Proc. Natl. Acad. Sci. USA, 90:6909 (1993); Erb et al., Proc. Natl. Acad. Sci. USA, 91:11422 (1994); Zuckermann et al., J. Med. Chem., 37:2678 (1994); Cho et al., Science, 261:1303 1993); Carrell et al., Angew. Chem. Int. Ed. Engl., 33:2059 (1994); Carell et al., Angew. Chem. Int. Ed. Engl., 33:2061 (1994); and in Gallop et al., J. Med. Chem., 37:1233 (1994). Libraries of compounds can be presented in solution (e.g., Houghten (1992) Biotechniques, 13:412-421), or on beads (Lam (1991) Nature, 354:82-84), chips (Fodor (1993) Nature, 364:555-556), bacteria (Ladner, U.S. Pat. No. 5,223,409), spores (Ladner, U.S. Pat. No. 5,223,409), plasmids (Cull et al. (1992) Proc. Natl. Acad. Sci. USA, 89:1865-1869) or on phage (Scott and Smith (1990) Science, 249:386-390; Devlin (1990) Science, 249:404-406; Cwirla et al. (1990) Proc. Natl. Acad. Sci. USA, 87:6378-6382; Felici (1991) J. Mol. Biol., 222:301-310; Ladner, supra.).

In some embodiments, the methods of the disclosure are used to screen “approved drugs”. An “approved drug” is any compound (which term includes biological molecules such as proteins and nucleic acids) which has been approved for use in humans by the FDA or a similar government agency in another country, for any purpose.

Compounds identified as having a beneficial effect (e.g. candidate therapeutic compounds), or in general a morphological effect (inhibitors, and in particular candidate therapeutic compounds, or enhancer of the redox active molecules), are referred to herein as lead compounds and can be selected and systematically altered, e.g., using rational design, to optimize binding affinity, avidity, specificity, or other parameters. Such optimization can also be screened for using the methods described herein. Thus, one can screen a first library of small molecules using the methods described herein, identify one or more compounds that are “hits” or “leads” (by virtue of, for example, their ability to alter the morphology, growth and/or survival of a test cell), and subject those hits to systematic structural alteration to create a second library of compounds (e.g., refined lead compounds) structurally related to the hit. The second library can then be screened using the methods described herein. A refined lead compound can be produced by modifying the lead compound to achieve (i) improved potency, (ii) decreased toxicity (improved therapeutic index); (iii) decreased side effects; (iv) modified onset of therapeutic action and/or duration of effect; and/or (v) modified pharmacokinetic parameters (absorption, distribution, metabolism and/or excretion). The lead compound could be, e.g., purified from natural sources or chemically synthesized. Modifications could be made directly to the lead compound, or refined lead compounds (e.g., derivatives) could be synthesized from suitable starting materials.

Accordingly, aspects of the disclosure relate to screening for compounds that inhibit the production of one or more redox-active molecules in a redox-active organism.

Aspects of the disclosure also may be used to treat infections caused by redox-active organisms. In some embodiments, one or more compounds that inhibit the production of a redox-active molecule in an organism (e.g., in a bacterium) may be used as an antibiotic against that organism. In some embodiments, one or more compounds that inhibit the production of a redox-active molecule in an organism (e.g., in a bacterium) may be used to weaken that organism (e.g., in an infection) and increase its susceptibility to treatment by one or more additional compounds.

As used herein, a redox-active organism is an organism (e.g., a unicellular organism) that produces a redox-active molecule as described herein. In some embodiments, redox-active organisms may be proteobacteria or actinobacteria. In some embodiments, redox-active organisms may be bacteria that include one or more soxR responsive genes selected from the group consisting of including transporters, oxygenases, dehydrogenases, putative acetyl/methyltransferases and LPS-P ribonucleases.

In some aspects, the disclosure relates to bacteria comprising SoxR binding sites in the promoter regions of target genes. In some embodiments, bacteria comprising SoxR binding sites in the promoter regions of target genes are useful for identifying candidate therapeutic compounds. In other aspects the bacteria comprising SoxR binding sites in the promoter regions of target genes are useful for identifying candidate compounds for enhancing power output of a microbial fuel cells that comprise the bacteria. Exemplary SoxR binding site containing bacteria are: Acidobacteria bacterium Ellin345; Acidovorax avenae subsp. citrulli AAC00-1; Acinetobacter baumannii ATCC 17978; Acinetobacter baumannii AYE; Acinetobacter baumannii; Acinetobacter sp. ADP1; Aeromonas hydrophila subsp. hydrophila ATCC 7966; Aeromonas salmonicida subsp. salmonicida A449; Agrobacterium tumefaciens str. C58; Arthrobacter aurescens TC1; Azoarcus sp. BH72; Bacillus anthracis str.' Ames Ancestor'; Bacillus anthracis str. Ames; Bacillus anthracis str. Sterne; Bacillus cereus ATCC 14579; Bacillus cereus E33L; Bacillus subtilis subsp. subtilis str. 168; Bacillus thuringiensis str. Al Hakam; Bacillus thuringiensis serovar konkukian str. 97-27; Bdellovibrio bacteriovorus HD100; Bordetella bronchiseptica RB50; Bordetella pertussis Tohama I; Burkholderia sp. 383 chromosome 2; Burkholderia ambifaria MC40-6 chromosome 2; Burkholderia cenocepacia AU 1054 chromosome 2; Burkholderia cenocepacia HI2424 chromosome 2; Burkholderia cenocepacia MC0-3 chromosome 2; Burkholderia cepacia AMMD chromosome 2; Burkholderia multivorans ATCC 17616 chromosome 2; Chromobacterium violaceum ATCC 12472; Citrobacter koseri ATCC BAA-895; Clavibacter michiganensis subsp. sepedonicus; Clostridium beijerinckii NCIMB 8052; Delftia acidovorans SPH-1; Desulfitobacterium hafniense Y51; Enterobacter sp. 638; Enterobacter sakazakii ATCC BAA-894; Erythrobacter litoralis HTCC2594; Escherichia coli 536; Escherichia coli APEC O1; Escherichia coli CFT073; Escherichia coli DH10B; Escherichia coli E24377A; Escherichia coli HS; Escherichia coli str. K-12 substr. MG1655; Escherichia coli O157:H7 EDL933; Escherichia coli O157: H7 str. Sakai; Escherichia coli SECEC SMS-3-5; Escherichia coli UT189; Frankia sp. Cc13; Frankia sp. EAN1pec; Hahella chejuensis KCTC 2396; Herpetosiphon aurantiacus ATCC 23779; Hyphomonas neptunium ATCC 15444; Idiomarina loihiensis L2TR; Janthinobacterium sp. Marseille; Kineococcus radiotolerans; SRS30216; Klebsiella pneumoniae subsp. pneumoniae MGH 78578; Lactobacillus casei ATCC 334; Lactobacillus sakei subsp. sakei 23K; Lactobacillus salivarius UCC118; Lactococcus lactis subsp. cremoris MG1363; Lactococcus lactis subsp. cremoris SK11; Lactococcus lactis subsp. lactis I11403; Maricaulis marls MCS10; Marinomonas sp. MWYL1; Mesorhizobium loti MAFF303099; Mycobacterium abscessus; Mycobacterium gilvum PYR-GCK; Mycobacterium sp. JLS; Mycobacterium sp. KMS; Mycobacterium sp. MCS; Mycobacterium smegmatis str. MC2 155; Mycoplasma penetrans HF-2; Myxococcus xanthus DK 1622; Nocardia farcinica IFM 10152; Novosphingobium aromaticivorans DSM 12444; Ochrobactrum anthropi ATCC 49188; Pelotomaculum thermopropionicum SI; Photobacterium profundum SS9; Polaromonas sp. JS666; Pseudoalteromonas atlantica T6c; Pseudomonas aeruginosa PAO1; Pseudomonas aeruginosa PA7; Pseudomonas aeruginosa UCBPP-PA14; Pseudomonas entomophila L48; Pseudomonas fluorescens Pf-5; Pseudomonas fluorescens PfO-1; Pseudomonas mendocina ymp; Pseudomonas putida F1; Pseudomonas putida GB-1; Pseudomonas putida KT2440; Pseudomonas putida W619; Pseudomonas stutzeri A1501; Ralstonia eutropha H16; Ralstonia metallidurans CH34; Renibacterium salmoninarum ATCC 33209; Rhizobium etli CFN 42; Rhizobium leguminosarum bv. viciae 3841; Rhodopseudomonas palustris BisB5; Saccharopolyspora erythraea NRRL 2338; Salinispora tropica CNB-440; Salmonella enterica subsp. arizonae serovar 62:z4,z23:-; Salmonella enterica subsp. enterica serovar Choleraesuis str. SC-B67; Salmonella enterica subsp. enterica serovar Paratyphi A str. ATCC 9150; Salmonella enterica subsp. enterica serovar Paratyphi B str. SPB7; Salmonella typhimurium LT2; Salmonella enterica subsp. enterica serovar Typhi str. CT18; Salmonella enterica subsp. enterica serovar Typhi Ty2; Shewanella amazonensis SB2B; Shewanella denitriflcans OS217; Shewanella frigidimarina NCIMB 400; Shewanella loihica PV-4; Shigella dysenteriae Sd197; Shigella flexneri 2a str. 2457T; Shigella flexneri 2a str. 301; Shigella flexneri 5 str. 8401; Shigella sonnei Ss046; Silicibacter pomeroyi DSS-3; Silicibacter sp. TM1040; Sinorhizobium medicae WSM419; Sinorhizobium meliloti 1021; Sorangium cellulosum'So ce 56′; Sphingopyxis alaskensis RB2256; Streptomyces avermitilis MA-4680; Streptomyces coelicolor A3(2); Thermobifida fusca YX; Trichodesmium erythraeum IMS101; Vibrio cholerae O1 biovar eltor str.; Vibrio cholerae O395; Vibrio harveyi ATCC BAA-1116; Vibrio harveyi ATCC BAA-1116; Vibrio parahaemolyticus RIMD 2210633; Vibrio vulnificus CMCP6; Vibrio vulnificus YJ016; Xanthomonas campestris pv. campestris str. 8004; Xanthomonas campestris pv. campestris str. ATCC 33913; Xanthomonas campestris pv. vesicatoria str. 85-10; Xanthomonas axonopodis pv. citri str. 306; Xanthomonas oryzae pv. oryzae MAFF 311018; and Zymomonas mobilis subsp. mobilis ZM4.

Aspects of the disclosure provide methods for treating certain bacterial infections. In some embodiments, compounds that are known or identified (e.g., using screening methods of the disclosure) as inhibitors of redox-active compounds may be administered to a subject at risk of infection or already infected by a redox-active organism as described herein. It should be appreciated that inhibitors may be compounds that inhibit (e.g., partially or completely reduce the level of activity of) one or more synthetic, transport, transformation, and/or regulatory steps of a redox-active compound produced by a redox-active organism.

In particular, inhibitors that can be administered to a subject to treat in the subject infections associated with a redox-active organism (treating inhibitors) are inhibitors that have been further selected through preclinical and clinical studies to assess the relevant efficacy, safety (pharmacovigilance), tolerability, pharmacokinetics, and pharmacodynamics in the subject before administration. Those tests and trials include but are not limited to in vitro and in vivo tests and studies, first-in-human-trials, Single Ascending Dose studies (SAD), Multiple Ascending Dose studies (MAD studies), trials designed to investigate any differences in absorption of the inhibitor by the body, caused by eating before the inhibitor is given and other trials established by the U.S. Food and Drug Administration and identifiable by a skilled person

In some embodiments, the inhibitor inhibits production of redox-active compounds in the bacteria of the infection. In some embodiments, the inhibitor inhibits secretion of redox-active compounds from the bacteria of the infection. In some embodiments, the inhibitor reduces levels of the redox-active compounds produced by the bacteria of the infection in the patient.

In some embodiments of the treatment methods, the bacterial infection is caused by a pathogen of the Actinobacteria or Proteobacteria phyla. In some embodiments, the bacterial infection is caused by a pathogen of the Pseudomonas genus. In some embodiments, the bacterial infection is caused by a Pseudomonas pathogen selected from the group consisting of: P. aeruginosa, P. oryzihabitans, P. fluorescens, and P. luteola. In other embodiments, the bacterial infection is caused by a pathogen of the Streptomyces genus. In other embodiments, the pathogen is resistant to beta-lactam antibiotics, penicillin, piperacillin, imipenem, tobramycin, or ciprofloxacin.

As used herein, treatment may be prophylactic (e.g., to prevent or reduce the risk of an infection) or therapeutic or curative. Accordingly, subjects to be treated may be subjects that are infected or subjects that are at risk of infection. Subjects at risk of infection may be immuno-compromised subjects or subjects that have a condition that makes them susceptible to infection by one or more organisms (e.g., bacterial pathogens) described herein. For example a subject at risk of infection may be a subject that has an HIV infection, AIDS, Cystic Fibrosis, or other disease or condition that causes an immunodeficiency. In some embodiments, a subject at risk of infection may be a subject that has been wounded (e.g., suffered a cut or other wound) or a subject that is undergoing or has undergone surgery. A subject having an infection may be a subject infected with one or more organisms described herein. An infection may be a systemic infection or a wound infection (e.g., at the site of a cut or abrasion, including for example, at the site of a surgical incision) or any other type of infection (for example, any infection where anaerobic conditions may exist or prevail at the site of infection).

Accordingly, a compound of the disclosure may be administered to any suitable subject prior to, during, or after infection (or prior to, during, or after exposure to a disease, condition, accident, or procedure that exposes the subject to a risk of infection).

The terms “treatment” and “treating” are intended to encompass also prophylaxis, therapy and cure. Accordingly, in one aspect, a treatment includes preventing or delaying or slowing the onset of an infection (e.g. the symptoms associated with an infection). In another aspect, a treatment includes treating (e g minimizing or reducing or slowing the development or reversing) an existing infection (e.g. the symptoms associated with the infection). In some embodiments, a treatment provides a cure for an infection. In some embodiments, a treatment reduces the severity or extent of an infection. A subject receiving this treatment may be any animal in need of such treatment, including primates, in particular humans, and other mammals such as equines, cattle, swine and sheep; and poultry and pets in general.

In some embodiments of the treatment methods, the patient is immunocompromised or is at risk of becoming immunocompromised. In certain embodiments, the patient suffers from cystic fibrosis or HIV/AIDS. There are a variety of situations known in the art in which an individual may be deemed at risk of for infection by a redox active bacterium. In some embodiments, the patient is a patient receiving cancer therapy, a low birth weight infant, an intra-abdominal surgery patient, an individual having a severe urinary tract infection, an intensive care unit patient receiving anti-infective agents, an individual having severe community-acquired pneumonia (CAP), a burn or trauma victim, an individual having Meningitis, or Cellulitis, a hospitalized patient receiving cytotoxic and immunosuppressive agent(s) and an individual having a chronic disease such as diabetes, heart failure, chronic renal failure, and hepatitis. These examples are not meant to be limiting and the treatment methods of the disclosure may be useful in any instance where an individual has or is at risk of infection by a redox active bacteria.

Accordingly, aspects of the disclosure comprise methods for treating bacterial infections by disrupting the biological synthesis, secretion, and/or transformation of redox-active compounds produced by the bacteria itself.

Some embodiments of the disclosure include inhibiting the biological synthesis (biosynthesis) of redox-active compounds. Targets of such inhibition include, but are not limited to, the genes and proteins comprising the associated biosynthetic pathways and any additional cellular components which affect the biosynthetic pathway. Additional targets include genes and proteins responsible for the modification of redox-active compounds. Such transformations include, but are not limited to, oxidations, reductions, glycosylations, phosphorylations, and alkylations of the redox-active compounds subsequent to their biosynthesis. Further targets include genes and proteins responsible for the transport (e.g., secretion) of redox-active compounds.

Accordingly, in some embodiments of the treatment methods, the inhibitor disrupts bacterial biosynthesis of a redox-active compound. In some embodiments, the inhibitor disrupts bacterial transformation of a redox-active compound subsequent to its bacterial biosynthesis. In some embodiments, the inhibitor disrupts bacterial secretion of a redox-active compound. In some embodiments, the inhibitor decreases the production or the utilization of shikimic acid in the bacteria. In some embodiments, the inhibitor decreases the production or utilization of and chorismic acid in the bacteria.

Certain embodiments of the present disclosure reduce bacterial levels of redox-active compounds by disrupting the bacteria's ability to regulate levels of these compounds. Targets of such disruptions include, but are not limited to, the genes and proteins that regulate bacterial biosynthesis, maintenance, transport, and expulsion of redox-active compounds. Further targets include the genes, proteins, and small molecules such as N-acyl-homoserine lactones which mediate quorum sensing among bacteria. Additional targets include, but are not limited to, genes and proteins associated with global regulation. A non-limiting example of such a global regulator of phenazine production in Pseudomonas aureofaciens is the highly conserved GacA/S two-component signal transduction system.

As used herein, the term redox-active compounds refers to organic (e.g., naturally occurring) redox-active molecules. Redox-active natural products include, but are not limited to those produced by the genera Streptomyces and Pseudomonas, including those redox-active natural products produced by P. aeruginosa, P. oryzihabitans, and P. luteola.

Redox-active natural products include, but are not limited to the phenazine class comprising those described by Laursen J B & Nielsen J Chem. Rev. 2004, 104, 1663-1685 and Turner J M & Messenger A J Adv. Microb. Physiol. 1986, 27, 210-275.

Phenazines are natural products substituted at different points around the heterocyclic core by various bacterial species. Many phenazines are pigmented. Modifications of the phenazine core structure may result in color changes that encompass a broad spectrum, ranging from the deep red of 5-methyl-7-amino-1-carboxyphenazinium betaine (aeruginosin A) to the lemon yellow of phenazine-1-carboxylic acid, to the bright blue of 1-hydroxy-5-methylphenazine (pyocyanin).

Specific phenazine natural products include, but are not limited to, pyocyanin, aeruginosin A, phenazine-1-carboxylic acid, 2-hydroxyphenazine-1-carboxylic acid, phenazine-1-carboxamide, 1-hydroxyphenazine, 1,6-dihydroxyphenazine and iodinin. Additional phenazine natural products include, but are not limited to, pyocyanin, aeruginosin A, phenazine-1-carboxylic acid, 2-hydroxyphenazine-1-carboxylic acid, phenazine-1-carboxamide, 1-hydroxyphenazine, 1,6-dihydroxyphenazine and iodinin, 1,6-dihydroxyphenazine, 1,6-dihydroxyphenazine-5-oxide, 1,6-dimethoxyphenazine, 1,8-dihydroxyphenazine, 1,8-dihyroxyphenazine-10-oxide, 1-hydroxy-6-methoxy phenazine, 1-hydroxy-8-aminophenazine, 1-hydroxyphenazine, 1-methoxyphenazine, 2,3,4-trihydroxyphenazine-1-carboxylic acid, 2,3,7-trihydroxyphenazine, 2,3,7-trihydroxyphenazine-1,6-dicarboxylic acid, 2,3-di-(1-methoxycarbonyl-6-phenazinyl)betaine, 2,3-dihydroxyphenazine, 2,3-dihydroxyphenazine-1-carboxylic acid, 2,6-dihydroxyphenazine-1-carboxylic acid, 2,9-dihydroxyphenazine-1-carboxylic acid, 2-hydroxyphenazine, 2-hydroxyphenazine-1-carboxylic acid, 2-hydroxyphenazine-1-carboxylic acid, 4,9-dihydroxyphenazine-1,6-dicarboxylic acid dimethyl ester, 4-hydroxyphenazine-1,6-dicarboxylic acid dimethyl ester, 5-deoxy iodinin, 6-(1-hydroxyethyl)phenazine-1-carboxylic acid methyl ester, 6-(1-methoxyethly)phenazine-1-carboxylic acid methyl ester, 6-(3-methyl-2-butenyl)phenazine-1-carboxylic acid, 6-hydroxymethyl-9-hydroxyphenazine-1-carboxylic acid, 6-hydroxyphenazine-1-carboxylic acid, 6-hydroxyphenazine-1-carboxylic acid, 9-hydroxyphenazine-1-carboxylic acid, aeruginosin A, aeruginosin B, aestivopheonin A, aestivopheonin B, aestivopheonin C, benthocyanin A, benthocyanin B, benthocyanin C, chloroaphine, D-alanyl griseoluteic acid, DC-86-M, di-(2-hydroxy-1-phenazinyl)methane, di-phenazithionin, DOB-41, endophenazine A, endophenazine B, endophenazine C, endophenazine D, esmeraldine, esmeraldine, griseoluteic acid, griseoluteic acid, griseolutein A, griseolutein B, hemipyocyanin, hemipyocyanin, hemipyocyanine, iodinin, LL-141352β, LL-141352α, lomofungin, methanophenazine, mycomethoxin A, mycomethoxin, mycomethoxin B, mycomethoxin B, myxin, oxychlororaphine, oxychlororaphine 5,10-dihydro derivative, PD 116,152, elagiomicin A, pelagiomicin B, pelagiomicin C, phenacein, phenainomycin, phenazine 1,6-dicarboxylic acid, phenazine-1,6-dicarboxylic acid, phenazine-1,6-dicarboxylic acid, phenazine-1,6-dicarboxylic acid dimethyl ester, phenazine-1-carboxylic, phenazine-1-carboxylic, phenazine-1-carboxylic acid, phenazioviridine, phenazostatin A, phenazostatin B, phenazostatin C, phencomycin, pyocyanin, quinovosyl esters, saphenic acid, saphenomycin, SB 212021, SB 212305, sendomycin A, sendomycin B, sephenamycin, tubermycin B, tubermycin B¹⁴,WS-9659A, Lavanducyanin, and WS-9659B.

Further examples of redox-active natural products include, but are not limited to the quinone class and their derivatives such as hydroquinones, semiquinones, anthraquinones and quinolones. Specific examples of quinone natural products include actinorhodin, pyrroloquinoline, and 2-heptyl-3-hydroxy-4-quinolone. Other examples include co-factors and primary metabolitses, including for example pyridines, nicotinic acids, nicotinamides, flavins or isoalloxazines, pterins, ascorbates, etc., or any combination thereof.

In some embodiments of the treatment methods, the redox-active compound is a phenazine. In certain embodiments, the phenazine is selected from the group consisting of pyocyanin, aeruginosin A, phenazine-1-carboxylic acid, 2-hydroxyphenazine-1-carboxylic acid, phenazine-1-carboxamide, 1-hydroxyphenazine, 1,6-dihydroxyphenazine and iodinin. In some embodiments, the redox-active compound is selected from the group consisting of a quinone, hydroquinone, semiquinone, anthraquinone and a quinolone. In some embodiments, the redox-active compound is actinorhodin.

As used herein, an “inhibitor” can be, for example, an antibody, an antisense oligonucleotide, an aptamer, a short interfering nucleic acid molecule (siRNA), a small molecule inhibitor, or any other inhibitor which specifically or selectively disrupts the synthesis, secretion, and/or transformation of bacterial redox-active compounds.

For example, inhibitors of naturally occurring redox-active compounds such as phenazines comprise molecules which disrupt any portion of the phenazine biosynthetic pathways. Phenazines are derived from shikimic acid and chorismic acid. Therefore, inhibitors of shikimic acid biosynthesis and utilization, such as glyphosate, will also inhibit phenazine biosynthesis Likewise, inhibitors of chorismic acid biosynthesis and utilization, such as (6S)-6-fluoroshikimate (Kerbarh O, Bulloch E M, Payne R J, Sahr T, Rébeillé F, Abell C. Biochem Soc Trans. 2005 August; 33(Pt 4):763-6) and those described elsewhere (Dias M V, Ely F, Palma M S, de Azevedo W F Jr, Basso L A, Santos D S Curr Drug Targets. 2007 March; 8(3):437-44) will inhibit phenazine biosynthesis.

It also should be appreciated that an inhibitor may be a compound that is isolated or identified through a screening assay described herein.

In some embodiments, methods are provided for administering to a patient having a bacterial infection an inhibitor of redox active compounds that sensitizes the bacteria in the patient to one or more standard antibiotic compounds.

In some embodiments, the inhibitors of redox active compounds can be administered in combination with one or more standard antibiotic compound. In other embodiments, the inhibitors of redox active compounds are administered in advance of the a standard antibiotic compounds. Exemplary standard antibiotic compounds include, but are not limited to: Aminoglycosides, such as Amikacin, Gentamicin, Kanamycin, Neomycin, Netilmicin, Streptomycin, Tobramycin, Paromomycin; Ansamycins such as Geldanamycin and Herbimycin; the Carbacephem, Loracarbef; Carbapenems such as Ertapenem, Doripenem, Imipenem, and Meropenem; Cephalosporins, such as Cefadroxil, Cefazolin, Cefalotin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefdinir, Cefotetan, and Cefepime; Glycopeptides such as Teicoplanin and Vancomycin; Macrolides such as Azithromycin, Clarithromycin, Dirithromycin, Erythromycin, Roxithromycin, Troleandomycin, Telithromycin, and Spectinomycin; Monobactams such as Aztreonam; Penicillins such as Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Penicillin G, Piperacillin, and Ticarcillin; Polypeptides such as Bacitracin, Colistin and Polymyxin B; Quinolones and Fluoroquinolones such as Ciprofloxacin, Enoxacin, Gatifloxacin, Levofloxacin, Lomefloxacin, Moxifloxacin, Norfloxacin, Ofloxacin, and Trovafloxacin; Sulfonamides such as, Mafenide, Prontosil, Sulfacetamide, Sulfamethizole, Sulfanilimide, Sulfasalazine, Sulfisoxazole, Sulfadiazine, Trimethoprim, and Trimethoprim-Sulfamethoxazole; Tetracyclines such as Demeclocycline, Doxycycline, Minocycline, Oxytetracycline, and Tetracycline; and others including Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin), and Tinidazole.

In aspects of the disclosure, inhibitors may be prepared and/or administered as sterlized preparations and/or in combination with one or more pharmaceutically acceptable carriers. Accordingly, aspects of the disclosure relate to pharmaceutical compositions comprising one or more inhibitor compounds described herein.

In particular, in some embodiments, compositions containing any of the therapeutic compounds herein described in an optional pharmaceutically acceptable carrier are provided. Thus, in a related aspect, the disclosure provides a method for forming a medicament that involves placing a therapeutically effective amount of the therapeutic agent in the pharmaceutically acceptable carrier to form one or more doses. The effectiveness of treatment or prevention methods of the disclosure can be determined using standard methods known in the art.

In some embodiments, the present disclosure provides pharmaceutically acceptable compositions, which comprise a therapeutically effective amount of one or more of the compounds described herein, formulated together with one or more pharmaceutically acceptable carriers (additives) and/or diluents. As described in detail, the pharmaceutical compositions of the present disclosure may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin; intravaginally or intrarectally, for example, as a pessary, cream or foam; sublingually; ocularly; transdermally; or nasally, pulmonary and to other mucosal surfaces.

As used herein, the term “pharmaceutically acceptable” means a non-toxic material that does not interfere with the effectiveness of the biological activity of the active ingredients. In particular, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Pharmaceutically-acceptable carriers include any physiologically acceptable material, composition or vehicle, such as a liquid or solid filler, diluent, excipient, or solvent encapsulating material, involved in carrying or transporting the subject compound from one organ, or portion of the body, to another organ, or portion of the body.

The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term “carrier” denotes an organic or inorganic ingredient, natural or synthetic, with which the active ingredient is combined to facilitate the application. The components of the pharmaceutical compositions also are capable of being co-mingled with the compounds of the present disclosure, and with each other, in a manner such that there is no interaction which would substantially impair the desired pharmaceutical efficacy.

In several embodiments, each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Examples of such formulations include, but are not limited to DMSO, 10 mM DMSO, 8% hydroxypropyl-beta-cyclodextrin in PBS, propylene glycol, etc.

In several embodiments, when administered, the therapeutic compositions of the present disclosure are administered in pharmaceutically acceptable preparations. Such preparations may routinely contain pharmaceutically acceptable concentrations of salt, buffering agents, preservatives, compatible carriers, and optionally other therapeutic agents.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The therapeutics of the disclosure can be administered by any conventional route, including injection or by gradual infusion over time. The administration may, for example, be oral, aerosol, intravenous, intraperitoneal, intramuscular, intracavity, subcutaneous, or transdermal.

Regardless of the route of administration selected, the compounds of the present disclosure, which may be used in a suitable hydrated form, and/or the pharmaceutical compositions of the present disclosure, are formulated into pharmaceutically-acceptable dosage forms by conventional methods known to those of skill in the art.

The compositions of the disclosure are administered in effective amounts. An “effective amount” is that amount of a inhibitor of redox active compounds that alone, or together with further doses of the same or other compound (e.g. an antibiotic), produces the desired response, e.g. eradicating or sensitizing the bacteria. In the case of treating a particular infection characterized by pathogenic activity of the redox active bacteria disclosed herein, the desired response is inhibiting the progression of the disease (infection). This may involve only slowing the progression of the disease temporarily, although more preferably, it involves halting the progression of the disease permanently. This can be monitored by routine methods or can be monitored according methods well known in the art. The desired response to treatment of the infection also can be delaying the onset or even preventing the onset of the infection.

Such amounts will depend, of course, on the particular infection or redox active bacterial pathogen being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The doses of inhibitors of redox active compounds administered to a patient can be chosen in accordance with different parameters, in particular in accordance with the mode of administration used and the state of the patient. Other factors include the desired period of treatment. In the event that a response in a patient is insufficient at the initial doses applied, higher doses (or effectively higher doses by a different, more localized delivery route) may be employed to the extent that patient tolerance permits.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present disclosure employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts. A daily, weekly, or monthly dosage (or other time interval) can be used.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required to achieve the desired therapeutic effect and then gradually increasing the dosage until the desired effect is achieved.

In general, a suitable daily dose of a compound of the disclosure will be that amount of the compound that is the lowest dose effective to produce a therapeutic effect. Such an effective dose will generally depend upon the factors described above. Generally doses of the compounds of this disclosure for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight.

If desired, the effective daily dose of the active compound may be administered as two, three, four, five, six or more sub-doses administered separately at appropriate intervals throughout the day, optionally, in unit dosage forms. In some embodiments, one or more compounds of the disclosure may be formulated for extended release.

While it is possible for a compound of the present disclosure to be administered alone, it is preferable to administer the compound as a pharmaceutical formulation (composition). In another aspect, the present disclosure provides pharmaceutically acceptable compositions which comprise a therapeutically-effective amount of one or more of the subject compounds, as described herein, formulated together with one or more pharmaceutically acceptable carriers (additives) and/or diluents. As described in detail herein, the pharmaceutical compositions of the present disclosure may be specially formulated for administration in solid or liquid form, including those adapted for the following: oral administration, for example, drenches (aqueous or non-aqueous solutions or suspensions), tablets, boluses, powders, granules, pastes for application to the tongue; parenteral administration, for example, by subcutaneous, intramuscular or intravenous injection as, for example, a sterile solution or suspension; topical application, for example, as a cream, ointment or spray applied to the skin, lungs, or oral cavity; or intravaginally or intrarectally, for example, as a pessary, cream or foam; sublingually; ocularly; transdermally; nasally; pulmonary or to other mucosal surfaces.

Compounds according to the disclosure may be formulated for administration in any convenient way for use in human or veterinary medicine, by analogy with other pharmaceuticals.

Compounds of the disclosure described herein can be combined with other therapeutic agents. The compounds of the disclosure and other therapeutic agent may be administered simultaneously or sequentially. When the other therapeutic agents are administered simultaneously they can be administered in the same or separate formulations, but are administered at the same time. The other therapeutic agents are administered sequentially with one another and with compounds of the disclosure, when the administration of the other therapeutic agents and the compounds of the disclosure is temporally separated. The separation in time between the administration of these compounds may be a matter of minutes or it may be longer.

The disclosure, in some aspects, relates to microbial fuel cells. In some embodiments, the microbial fuel cells comprise redox active bacteria with enhanced electron transfer capabilities. These redox active bacteria typically grow as biofilms attached to surface of electrodes of the microbial fuel cells and produce electron transfer mediators to facilitate power output. In some embodiments, the redox active bacteria produce enhanced levels of electron transfer mediators that mediate the reduction of iron oxide in the fuel cells. In some embodiments, the redox active bacteria of the fuel cells produce enhanced levels of phenazine compounds. In some embodiments, the phenazine compounds function as electron transfer mediators in the fuel cell.

In some embodiments, the microbial fuel cells comprise redox active bacteria that are Actinobacteria or Proteobacteria. In some embodiments, the redox active bacteria are of a Pseudomonas species, optionally wherein the Pseudomonas species is selected from P. aureofaciens, P. fluorescens, P. chlororaphis, and P. syriangeae. In certain embodiments, the fuel cell comprises redox-active bacteria that have a mutation in any gene, the inactivation of which results in increased production of phenazines. In some embodiments, the mutation is a deletion of the SoxR locus. In certain embodiments, the fuel cell comprises redox active bacteria having an inactivating mutation (e.g., insertion, inversion, deletion) in the mexGHI-opmD operon. In some embodiments, the mutation is a deletion of the mexGHI-opmD operon.

In some aspects, the disclosure provides methods for identifying candidate bacteria for enhancing power output of a microbial fuel cell. The methods involve (i) mutagenizing a test bacterial colony; (ii) determining morphology of the test bacterial colony; and (iii) comparing the morphology of the test bacterial colony to a reference, wherein the comparison is indicative of whether or not the test bacterial colony is comprised of a candidate bacteria for enhancing power output of a microbial fuel cell. The mutagenesis, which may be specific or random, may be performed, for example, by use of a suitable physical or chemical mutagenizing agent known in the art. Furthermore, the mutagenesis may be performed by use of any combination of these mutagenizing agents. Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues.

In some aspects, the disclosure provides methods for identifying a candidate compound for enhancing power output of a microbial fuel cell. The methods involve (i) contacting a redox active bacterial colony with a test agent; (ii) determining morphology of the redox active bacterial colony; and (iii) comparing the morphology of the redox active bacterial colony to a reference, wherein the comparison is indicative of whether or not the test agent is a candidate compound for enhancing power output of a microbial fuel cell. In some embodiments, the methods also involve acquiring a digital image of the colony, optionally wherein the digital image is acquired through a microscope. In certain embodiments, the morphological parameter is determined from the digital image.

In some embodiments of the foregoing methods, the microbial fuel cell comprises redox active bacteria that are Actinobacteria or Proteobacteria. In some embodiments, the redox active bacteria are of a Pseudomonas species, optionally wherein the Pseudomonas species is selected from P. aureofaciens, P. fluorescens, and P. chlororaphis.

In view of the foregoing, the inhibitors or enhancer of redox-active compounds herein described can be provided as a part of systems to perform any assay, including any of the assays described herein or a treatment of an infection. The systems can be provided in the form of kits of parts.

In a kit of parts, an inhibitor of a redox active compound and other reagents suitable to perform the assay can be comprised in the kit independently. Various inhibitor can be included in one or more compositions, and each inhibitor can be in a composition together with a suitable vehicle.

Additional components can include an antibiotic compound and other reagents identifiable by a skilled person upon reading of the present disclosure.

In some embodiments, the components of the kit can be provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

### Examples

The following examples illustrate exemplary applications based at least in part on the surprising findings of bioinformatic analysis coupled with gene expression studies in P. aeruginosa and Streptomyces coelicolor that revealed that the majority of SoxR regulons in bacteria lack the genes required for stress responses despite the fact that many of these organisms still produce redox-active small molecules. This finding indicated that redox-active pigments play a role independent of oxidative stress. Further, these compounds were shown to have profound effects on the structural organization of colony biofilms in both P. aeruginosa and S. coelicolor. Accordingly, aspects of the disclosure provide methods for identifying inhibitors of redox-active compounds and methods for treating subjects infected with redox-active organisms.

### Example 1

### Materials and Methods

Quantitative real time PCR: Streptomyces coelicolor A3(2) strains were grown on R5⁻ plates that were overlain with cellophane membranes (Spectrum Laboratory Products, Inc.) (J. Huang, C. J. Lih, K. H. Pan, S. N. Cohen, Genes and Development 15, 3183 (2001)). Plates were incubated at 30° C. for three days to allow pigment production. Then cells were treated with RNAProtect Bacteria Reagent (Qiagen) for 5 min at room temperature, scraped off the cellophane membrane and centrifuged for 10 min at 5000 g. Total RNA was extracted from the cell pellets using the RNeasy Plant Mini Kit (Qiagen) according to the manufacturer's instructions, including the optional DNase treatment. cDNA was generated with iScript (BioRad) and served as template for quantitative RT-PCR (Real Time 7300 PCR Machine, Applied Biosystems) using the Sybr Green detection system (Applied Biosystems). Samples were assayed in triplicate. The signal was standardized to SCO4548 (S. Mehra et al., Journal of Industrial Microbiology and Biotechnology 33, 159 (2006)) using the following equation: Relative expression=2^{(CTstandard-CTsample)}, where CT (cycle time) was determined automatically by the Real Time 7300 PCR software (Applied Biosystems). Primers (Integrated DNA Technologies) for Q-RT-PCR were designed using Primer3 software (S. Rozen, H. Skaletsky, Methods Mot Biol 132, 365 (2000)).

Criteria for primer design were a melting temperature of 60° C., primer length of ˜20 nucleotides, and an amplified PCR fragment of 100 base pairs. The following primers were used:

SCO1697:
5′-tgcctcagattccagagaaga-3′
and
5′-tcagacccttggactcgtaga-3′
SCO2478:
5′-acaccgtctccttccacaac-3′
and
5′-ctggtcgagcatcgtcttg-3′
SCO2479:
5′-agatcgtcgcgacctgtg-3′
and
5′-ccctggggtacacctgct-3′
SCO4266:
5′-gatgggcatcctccagttc-3′
and
5′-cgttcttcgcgtactgcac-3′
SCO4548:
5′-agatcttcgagctcaacaagg-3′
and
5′-gggcatctccatgatcca-3′

Colony morphology assay: colony morphology assay was performed with Pseudomonas aeruginosa and Streptomyces coelicolor as described herein below:
a) Pseudomonas aeruginosa PA 14: Strains were grown in LB medium to late exponential phase, then 10 μl were spotted onto 1% agar plates containing 10 g/L Tryptone Broth, supplemented with 40 μg/l Congo Red and 20 μg/l Coomassie Blue as described previously (L. Friedman, R. Kolter, Mot Microbial 51, 675 (2004)). Plates were incubated at room temperature. Colonies were imaged using an Epson Perfection 2400 Photo Scanner at 600 dpi resolution, Nikon stereomicroscope or a digital microscope (Proscope).
b) Streptomyces coelicolor A3(2): 10 μl of a spore suspension in water was spotted onto plates with R5-medium. Plates were incubated at room temperature for six days. Colonies were imaged using a Nikon stereomicroscope.

Phenazine extraction from agar plates: Biological triplicate cultures of the wild type and each mutant were grown up overnight in LB. OD500 was normalized using spent supernatant so that all colonies would be spotted using the same cell density. Cultures were spotted three times onto an agar miniplate (10 ml of 1% tryptone, 1% agar in a 60×15 mm plate) and incubated at room temperature for 6 days. Cells were scraped off the agar surface with a spatula. Agar was submerged in 10 ml chloroform in a glass jar and agitated for 30 minutes. The chloroform was transferred to a brown glass vial and the sample was dried down under a stream of nitrogen gas. The solid was resuspended in 1 ml acetonitrile and filtered. 100 μl were loaded onto the HPLC and analyzed as described previously (L. E. Dietrich, A. Price-Whelan, A. Petersen, M. Whiteley, D. K. Newman, Mol Microbial 61, 1308 (2006)).

### Example 2

### Redox-Active Organisms

The opportunistic pathogen Pseudomonas aeruginosa releases phenazines, redox-active antibiotics (D. V. Mavrodi, W. Blankenfeldt, L. S. Thomashow, Annu Rev Phytopathol 44, 417 (2006). A. Price-Whelan, L. E. Dietrich, D. K. Newman, Nat Chem Biol 2, 71 (2006)). Historically, attention has focused on their toxicity in bacteria and eukaryotes, which arises from the production of superoxide (S. Mahajan-Miklos, M. W. Tan, L. G. Rahme, F. M. Ausubel, Cell 96, 47 (1999); and M. Mazzola, R. J. Cook, L. S. Thomashow, D. M. Weller, L. S. Pierson, 3rd, Applied and Environmental Microbiology 58, 2616 (1992)). More recently, however, it has been recognized that these compounds have diverse physiological functions, particularly under oxygen-limited conditions (A. Price-Whelan, L. E. Dietrich, D. K. Newman, Nat Chem Biol 2, 71 (2006); M. E. Hernandez, A. Kappler, D. K. Newman, Appl Environ Microbiol 70, 921 (2004); Y. Wang, D. K. Newman, Environ. Sci. Technol. 42, 2380 (2008); and M. E. Hernandez, D. K. Newman, Cell Mol Life Sci 58, 1562 (2001)).

The blue phenazine pyocyanin is an intercellular signal that triggers a specific response in P. aeruginosa, with only 22 genes up-regulated, including the complete SoxR regulon (L. E. Dietrich, A. Price-Whelan, A. Petersen, M. Whiteley, D. K. Newman, Mol Microbiol 61, 1308 (2006)). The transcription factor SoxR is well-characterized in the enteric bacteria Escherichia coli and Salmonella enterica serovar Typhimurium as a stress response regulator. In these bacteria SoxR activates the transcription factor SoxS, that controls genes involved in the removal of superoxide and nitric oxide and protection from organic solvents and antibiotics. That SoxR-regulated genes were triggered by pyocyanin was therefore initially not surprising, as this would be consistent with the conventional view of phenazines as toxic compounds (S. I. Liochev, L. Benov, D. Touati, I. Fridovich, Journal of Biological Chemistry 274, 9479 (1999); P. J. Pomposiello, B. Demple, Journal of Bacteriology 182, 23 (2000); and I. R. Tsaneva, B. Weiss, Journal of Bacteriology 172, 4197 (1990)).

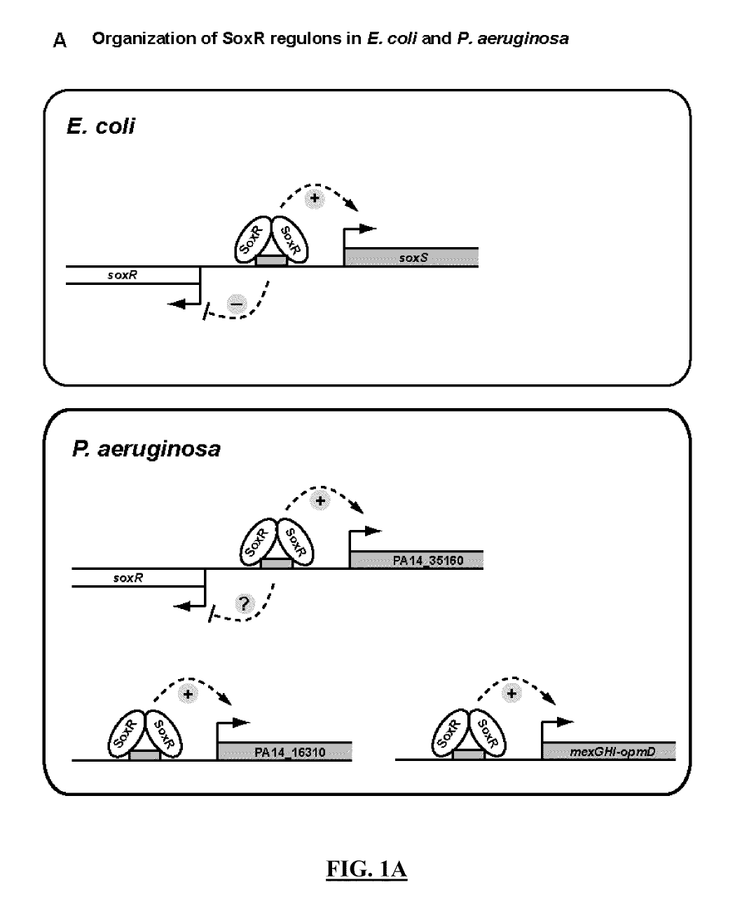

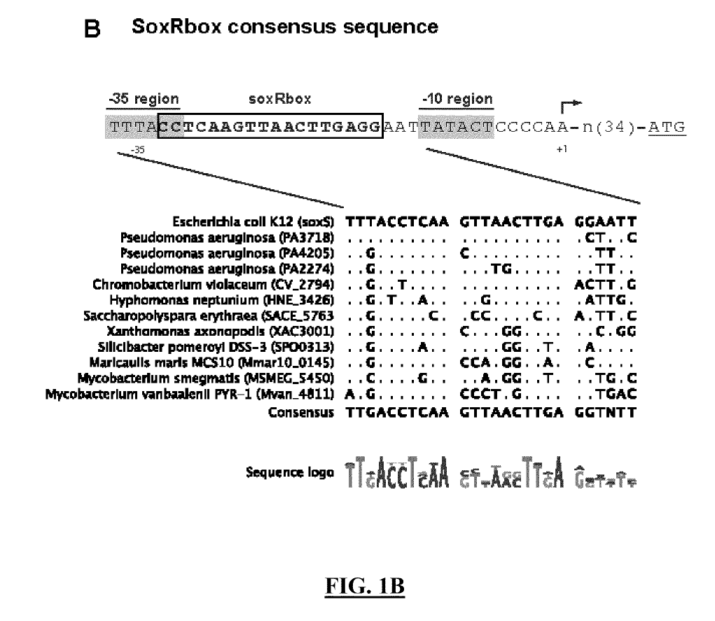

According to aspects of the disclosure, recent studies of the SoxR regulons in pseudomonads indicate an alternative role to the E. coli SoxR/S paradigm. First, superoxide is not the sole activator of SoxR, as P. aeruginosa pyocyanin also induces the expression of its regulon under anoxic conditions (L. E. Dietrich, A. Price-Whelan, A. Petersen, M. Whiteley, D. K. Newman, Mol Microbiol 61, 1308 (2006)). Second, SoxRs from Pseudomonas putida (W. Park, S. Pena-Llopis, Y. Lee, B. Demple, Biochemical and Biophysical Research Communications 341, 51 (2006)) and P. aeruginosa (L. E. Dietrich, A. Price-Whelan, A. Petersen, M. Whiteley, D. K. Newman, Mol Microbiol 61, 1308 (2006); K. Kobayashi, S. Tagawa, Journal of Biochemistry (Tokyo) 136, 607 (2004); and M. Palma et al., Infection and Immunity 73, 2958 (2005)) do not control any of the genes typically involved in superoxide resistance and detoxification, rather, SoxR from P. aeruginosa upregulates expression of two transporters and a putative monooxygenase (FIG. 1A). Third, P. aeruginosa soxR mutants show no decrease in resistance to superoxide, unlike E. coli soxR mutants (M. Palma et al., Infection and Immunity 73, 2958 (2005)).

According to aspects of the disclosure, redox-active signaling molecules, such as phenazines, also control other aspects of microbial behavior.

The distribution of the E. coli-type oxidative stress response was investigated by performing a BLAST search for SoxR and SoxS in the bacterial domain. SoxR was found in sequences from 165 strains in the phyla Proteobacteria and Actinobacteria (FIG. 1A), 123 of which come from completed genomes. The occurrence of SoxS was restricted to the family Enterobacteriaceae. To identify alternative SoxR targets in non-enterics we searched all available complete bacterial genomes (616) for the presence of soxRboxes (i.e., SoxR binding sites in the promoter regions of target genes) using a position weight matrix (PWM) derived from the soxRbox sequences of 12 diverse SoxR-containing bacteria (FIG. 1B). This PWM permits statistically robust predictions of SoxR binding to a soxRbox. 121 of the 123 soxR-containing genomes contain soxRboxes, and soxRboxes have also been found in 27 genomes (19 were Firmicutes) that do not contain a soxR homolog. The results of Applicants' analysis (Table 1 and http://soxRbox.mit.edu) were consistent with gene expression studies made in the Gram-negative bacteria E. coli, S. enterica (P. J. Pomposiello, B. Demple, Journal of Bacteriology 182, 23 (2000)), P. aeruginosa (L. E. Dietrich, A. Price-Whelan, A. Petersen, M. Whiteley, D. K. Newman, Mol Microbiol 61, 1308 (2006); K. Kobayashi, S. Tagawa, Journal of Biochemistry (Tokyo) 136, 607 (2004); and M. Palma et al., Infection and Immunity 73, 2958 (2005)), and Agrobacterium tumefaciens (W. Eiamphungporn, N. Charoenlap, P. Vattanaviboon, S. Mongkolsuk, Journal of Bacteriology 188, 8669 (2006)), validating our search algorithm. The results of Applicants' analysis are summarized in Table 1 described in the Appendix A enclosed in the present disclosure and forming integral part of the same.

In particular, in Table 1, Applicants' SoxRbox analysis is summarized. 616 bacterial genomes were analyzed for the presence of soxRbox elements. Strains containing soxRbox elements are listed. Site energy was calculated such that the site energy equals 0 for the consensus site and is greater than 0 for all other sites (C. T. Brown and C. G. Callan, Jr., Proc Natl Acad Sci USA 101 (8), 2404 (2004)).

Sequence is the soxRbox sequence for a given site. The operon position indicates whether the adjacent gene/operon is located 3′ (left) or 5′ (right) of the soxRbox, with respect to the top strand. Distance to start is the distance from the soxRbox to the start of the predicted operon. Orientation is the orientation of the soxRbox element, using the −35 region as the start of the soxRbox motif. Annotations for soxRbox genes can be found at http://soxRbox.mit.edu. in the version published at the filing date of the present application.

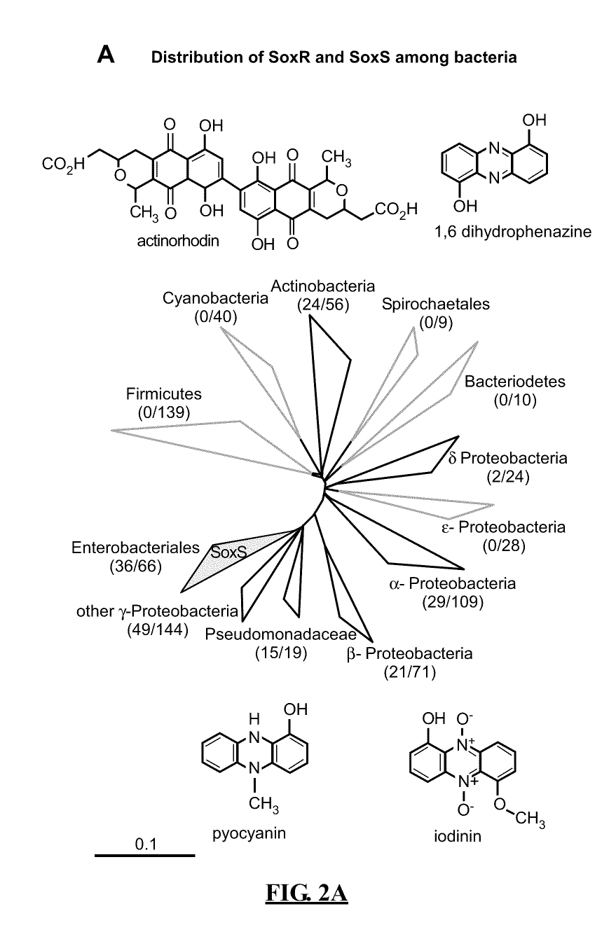

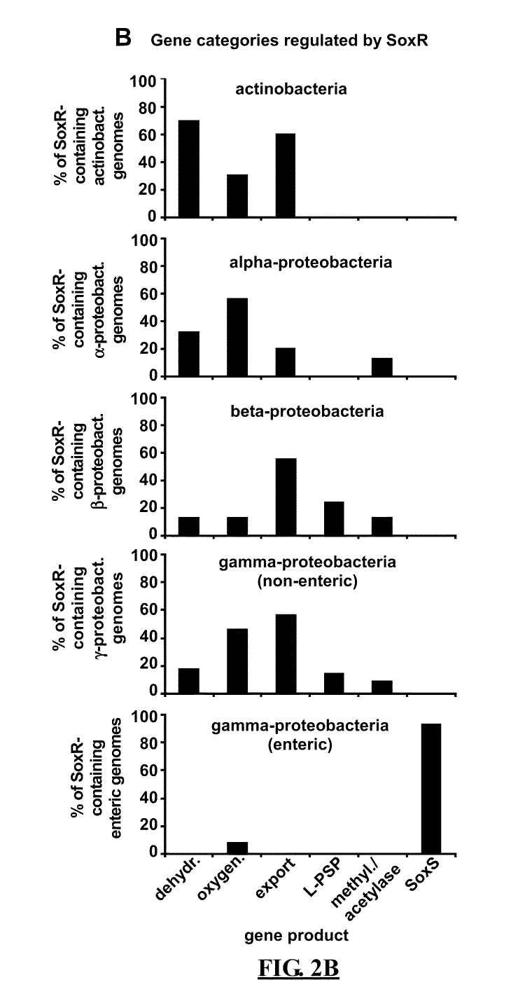

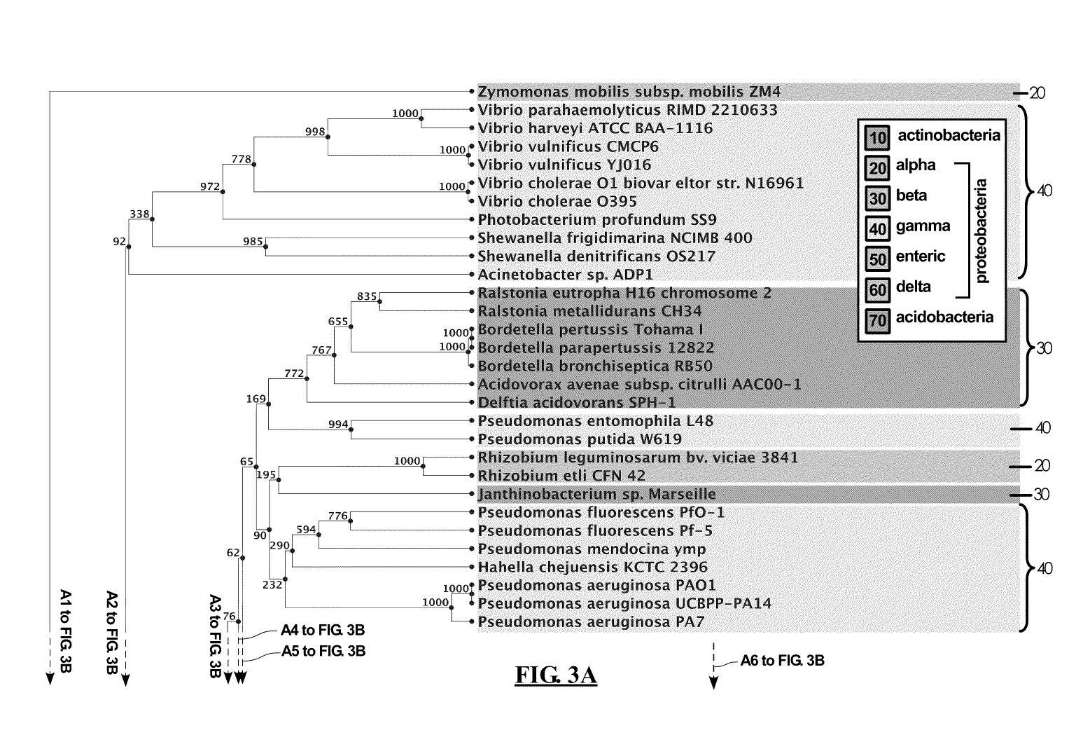

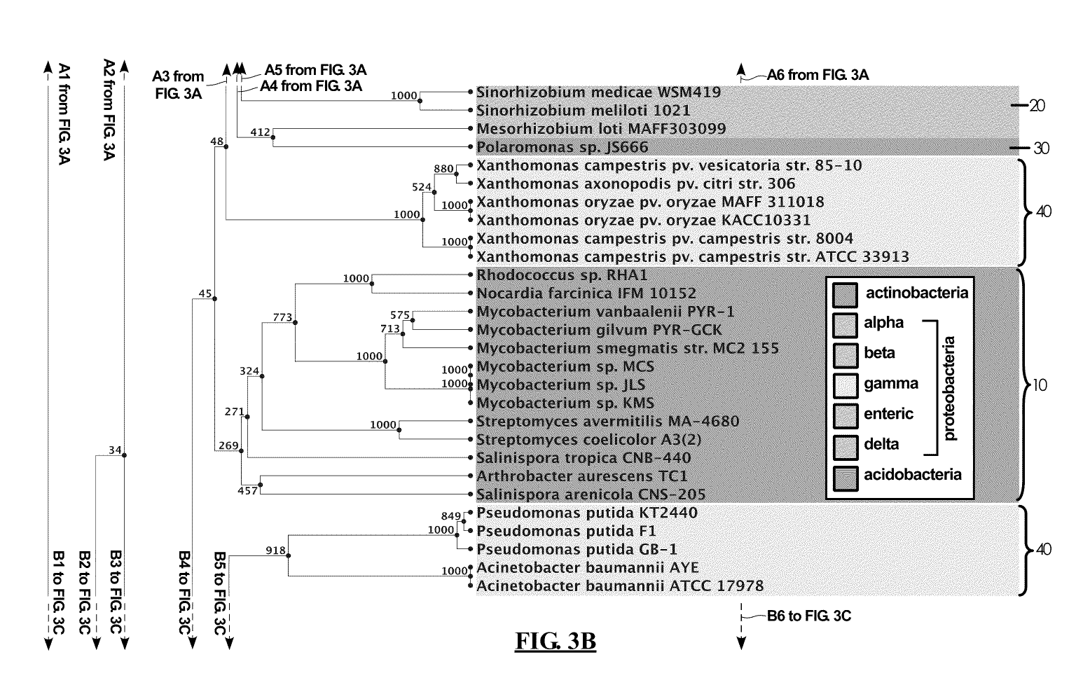

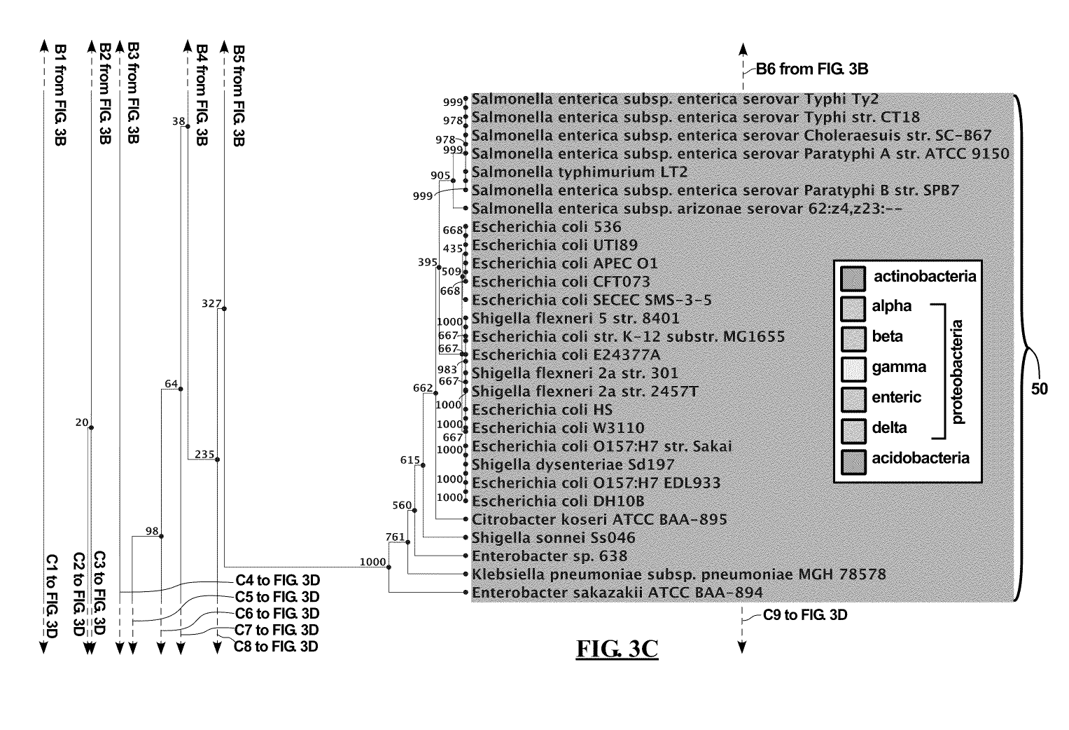

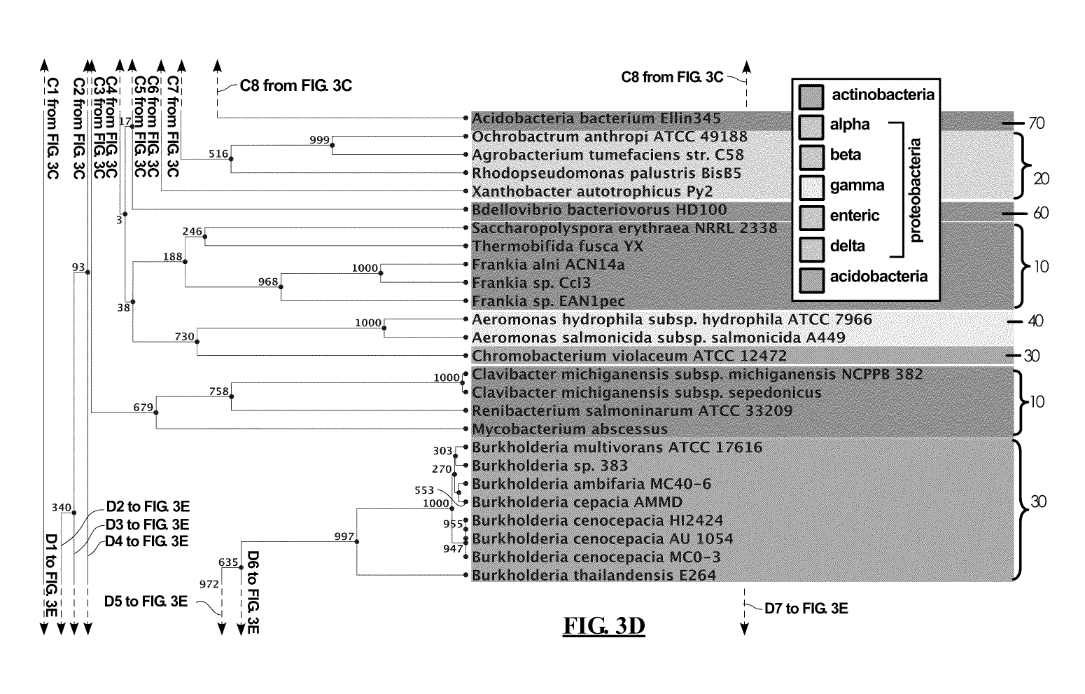

The organization found in E. coli (FIG. 1), with one soxRbox upstream of soxS and no other soxRboxes in the genome, occurred only in enterics (27 genomes) (FIG. 2B). Two enterics contained an additional soxRbox upstream of putative dioxygenases. The remaining organisms contained one or more soxRboxes upstream of genes other than soxS. These SoxR target genes fell into five main categories, including transporters, oxygenases, dehydrogenases, putative acetyl/methyltransferases and LPS-P ribonucleases, all of which are potentially involved in the transformation or transport of small molecules, such as antibiotics (E. Cundliffe, Annu Rev Microbiol 43, 207 (1989)). The occurrence of SoxR upstream of soxS in enterics thus appears to be an evolutionary exception confirmed by the unique branching of the enteric orthologs on a SoxR phylogenetic tree (FIG. 3).

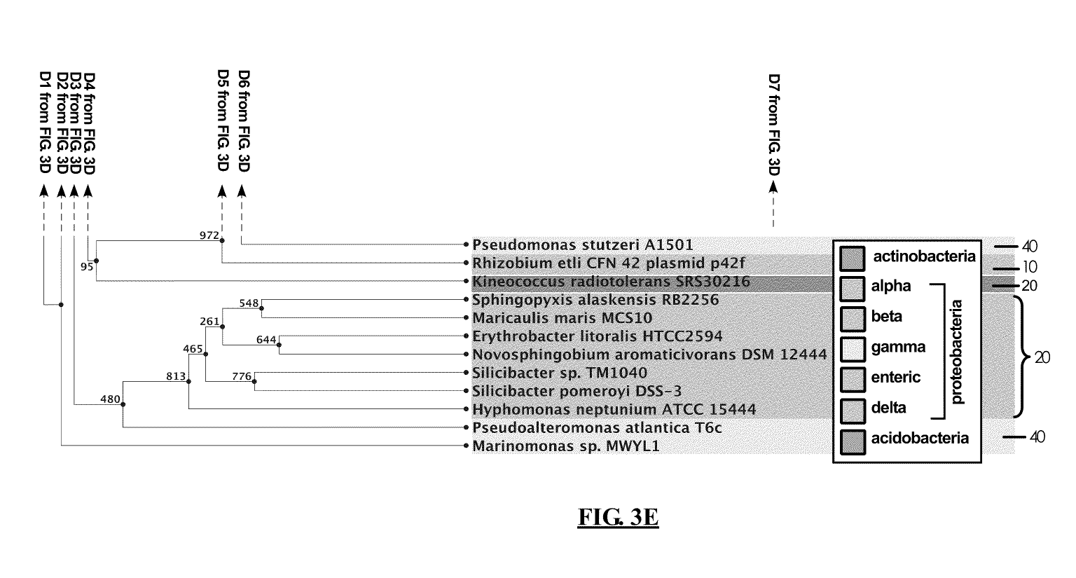

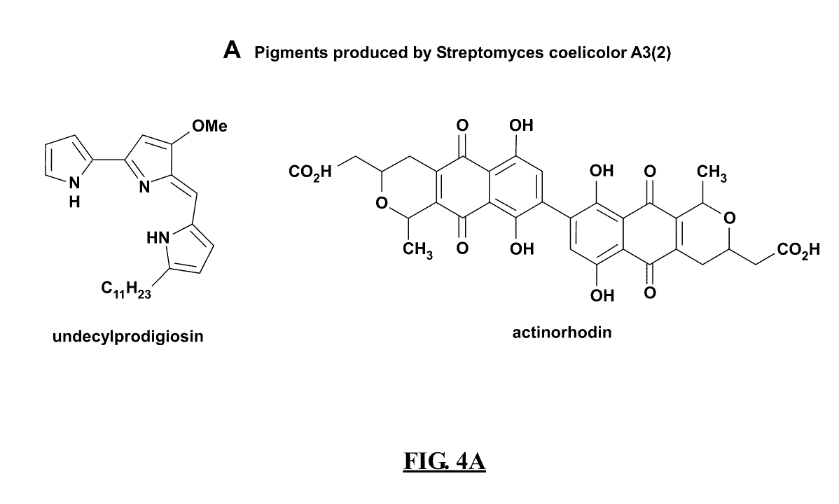

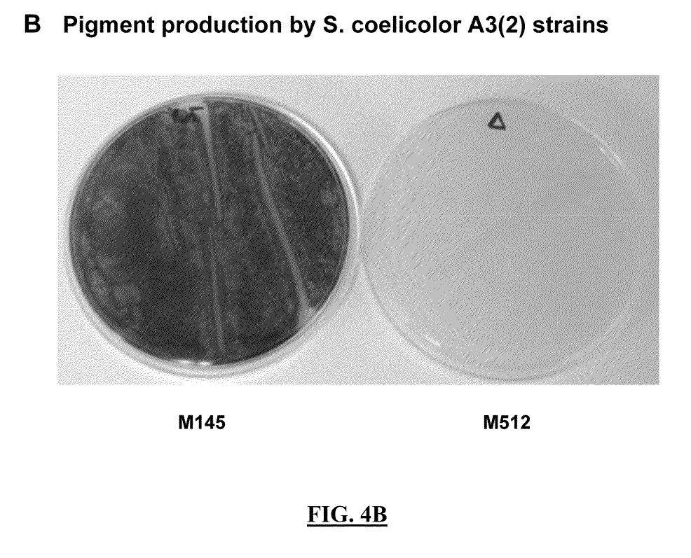

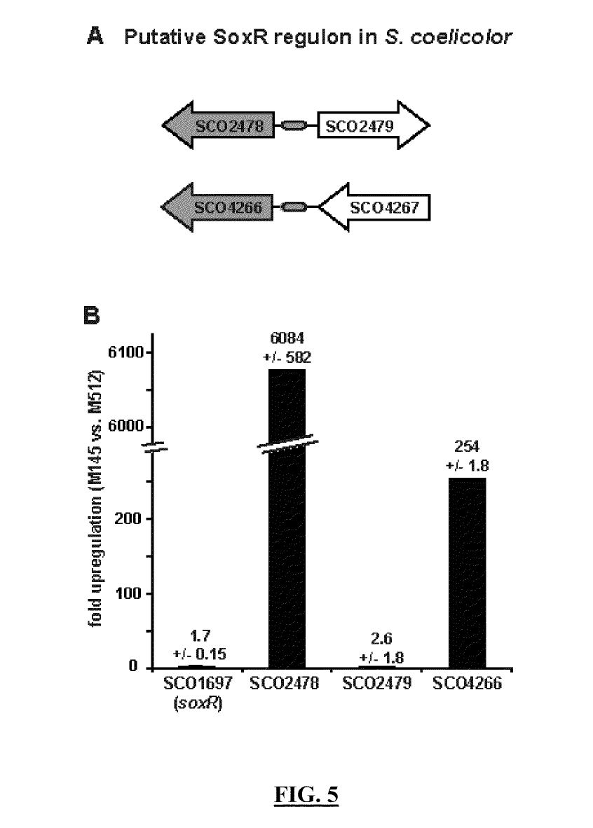

Given that many of the bacteria that contain soxRboxes are producers of redox-active antibiotics (J. M. Turner, A. J. Messenger, Adv Microb Physiol 27, 211 (1986) including species of Actinobacteria, delta-Proteobacteria, alpha-Proteobacteria, beta-Proteobacteria, Pseudomonadaceae, Enterobacteriales, and other gamma-Proteobacteria (FIG. 2A), it seems reasonable that SoxR may have evolved to regulate their transport and/or turnover. The Gram-positive actinomycete Streptomyces coelicolor A3(2) was used to test whether the SoxR regulon is upregulated in response to endogenous small molecules because members of this phylum are widely recognized as important sources of antibiotics (D. A. Hopwood, Streptomyces in Nature and Medicine. The Antibiotic Makers., Oxford University Press (2007)). S. coelicolor A3(2) produces the blue pigment actinorhodin and the red undecylprodigiosin (FIG. 4) (K. F. Chater, Philos Trans R Soc Lond B Biol Sci 361, 761 (2006)). Based on the analysis, a SoxR regulon was predicted comprising two genes for S. coelicolor A3(2), encoding putative redox enzymes (FIG. 5A). Expression of these genes was compared between the wild type (strain M145), and a mutant that does not produce the two pigments (strain M512) (B. Floriano, M. Bibb, Mol Microbiol 21, 385 (1996)). Both predicted SoxR-regulated genes were significantly upregulated in the wild type relative to the pigment-null mutant (˜250-6,000 fold) as determined by quantitative RT-PCR (FIG. 5B), confirming that pigment production stimulated gene expression via SoxR. Hence, the primary function of SoxR in S. coelicolor, as in P. aeruginosa, is not to activate a response to superoxide but to mediate a response to endogenous pigments.

### Example 3

### Red-Ox Active Molecules

Recently, phenazines were shown to be terminal signals in P. aeruginosa's quorum sensing cascade (L. E. Dietrich, A. Price-Whelan, A. Petersen, M. Whiteley, D. K. Newman, Mol Microbiol 61, 1308 (2006)). The importance of quorum sensing for the coordination of many bacterial communities is well established (D. G. Davies et al., Science 280, 295 (1998)). Moreover, a phenazine-dependent effect on biofilm formation has been reported in P. aureofaciens (V. S. Maddula, Z. Zhang, E. A. Pierson, L. S. Pierson, 3rd, Microbial Ecology 52, 289 (2006)).

According to aspects of the disclosure, redox-active pigments act as signals to modulate the structural organization of cellular communities.

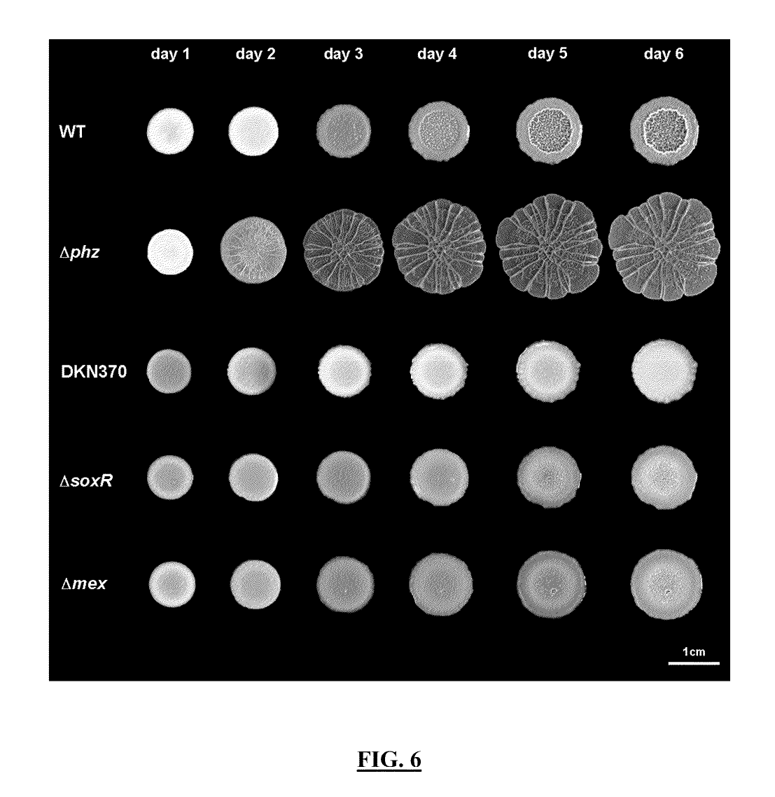

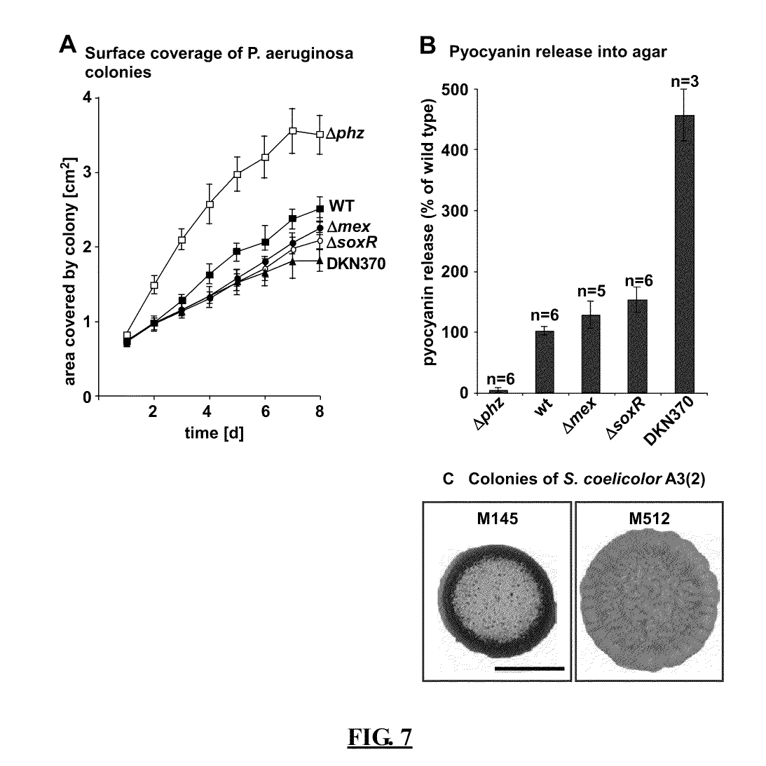

To investigate the effect of extracellular pigments on community development, P. aeruginosa PA14 was used. 10 μl aliquots of late exponential-phase cultures were spottedonto agar plates and incubated at room temperature for eight days. Under these conditions, wild type cells initially formed smooth colonies (FIG. 6). After four days of incubation, the colonies began to wrinkle and reached a maximum area of approximately 2.5 cm² (FIGS. 6 and 7A). Strikingly, the phenazine-null mutant formed severely wrinkled colonies within two days of incubation (FIG. 6), which subsequently flattened and spread to approximately 3.5 cm² (FIG. 7A). By contrast, a mutant that overproduced pyocyanin (DKN370) remained smooth and compact (FIG. 6). These results demonstrated a role for phenazines in controlling bacterial colony size and structure.

Phenazines are diffusible molecules that may influence phenotype over distance. Indeed, adding pyocyanin to the growth medium (FIG. 8A) or spotting the phenazine overproducer next to the phenazine deletion mutant (FIG. 8B) resulted in the formation of smooth compact colonies. The role of SoxR in mediating the effect of phenazines on colony was tested morphology by making a SoxR deletion mutant. However, the mutant behaved similarly to the pyocyanin overproducer and colonies remained smooth for 4 days (FIG. 6). As for the overproducer, ΔsoxR released more pyocyanin into the agar than the wild type (FIG. 7B). There thus appears to be a direct correlation between pyocyanin release and colony smoothness.

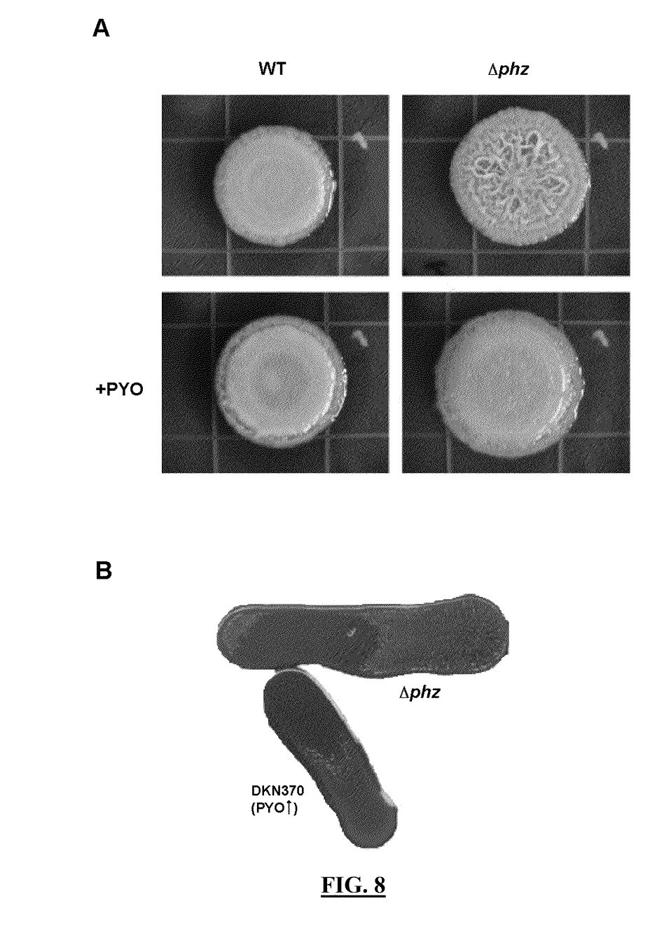

To further analyze the ΔsoxR phenotype, P. aeruginosa mutants disrupted in the SoxR target genes PA14_{—}35160 (encoding a putative monooxygenase), mexGHI-opmD (encoding a resistance-nodulation-cell division (RND) efflux pump) and PA14_{—}16310 (encoding a major facilitator superfamily (MFS) transporter) were tested. Deletions of PA14_{—}35160 and PA14_{—}16310 did not affect colony morphology; however, the loss of mexGHI-opmD produced a phenotype that looked like the ΔsoxR mutant, i.e. wrinkling was slow (FIG. 6), and was accompanied by a slightly elevated pyocyanin release (FIG. 7B). By contrast, the release of the yellow phenazine-1-carboxylate (PCA) and an unidentified red phenazine (possibly 5-methyl-PCA), decreased by 10% and 60%, respectively, in the mexGHI-opmD mutant relative to the wild type, indicating that mexGHI-opmD is a general phenazine transporter. Antibiotic biosynthetic genes are often found adjacent to their cognate transporter (K. Tahlan et al., Mol Microbiol 63, 951 (2007)), so it is interesting to note that the mexGHI-opmD operon is clustered with the phenazine biosynthetic genes phzM, phzA1-G1 and phzS (FIG. 9A).

PhzA1-G1 synthesizes the yellow phenazine PCA, and PhzM methylates PCA to yield the red phenazine 5-methyl-PCA, which is then hydroxylated by PhzS to form pyocyanin. Transposon insertion mutants in mexI and opmD of P. aeruginosa PAO1 are known to accumulate an unidentified toxic compound that causes an elongated lag phase in planktonic cultures (S. Aendekerk et al., Microbiology 151, 1113 (2005)). A similar phenotype was found in P. aeruginosa PA14 (FIG. 9B), which is probably caused by an intracellular accumulation of phenazines. These experiments showed that SoxR target genes do not directly influence colony development; instead, SoxR regulates the efflux of phenazines via the RND transporter MexGHI-OpmD. Although yellow PCA and red phenazine are retained in the mexGHI-opmD mutant, the release of pyocyanin indicates an alternative efflux mechanism favoring pyocyanin. Compensatory changes in expression of RND efflux pumps are well known to occur in P. aeruginosa (X. Z. Li, N. Barre, K. Poole, J Antimicrob Chemother 46, 885 (2000)).

### Example 4

### Redox-Active Molecules and Morphology of Redox Active Organisms

To determine whether the phenotypic effects of pigment production observed for P. aeruginosa were unique to this organism or more generalizable, analogous experiments were performed with S. coelicolor A3(2). As for P. aeruginosa, a pigment-defective mutant of S. coelicolor adopted a more wrinkled morphology than the respective wild type (FIG. 7C). The mechanisms whereby pigments control colony morphology are not understood, but are likely to be complex. For P. aeruginosa PA14, pyocyanin affects the expression of at least 35 genes other than those in the SoxR regulon (L. E. Dietrich, A. Price-Whelan, A. Petersen, M. Whiteley, D. K. Newman, Mol Microbiol 61, 1308 (2006)), and has profound effects on the cell's physiology, including the redox state of the intracellular NAD(H) pool (A. Price-Whelan, L. E. Dietrich, D. K. Newman, J Bacteriol (2007)).

Any number of these effects may contribute, both directly and indirectly, to the ultimate architectures observed. One component that is likely involved is extracellular polysaccharide (EPS). Congo Red, a constituent of the agar used in the experiments shown in FIG. 6, is known to bind the gluose-rich exopolysaccharide PEL (L. Friedman, R. Kolter, Mol Microbiol 51, 675 (2004)). Because the phenazine-null mutant is bright red, whereas the pyocyanin overproducer is pale there is an inverse relationship between phenazine and PEL production (FIG. 6). How phenazines affect the pel genes, and how such changes in EPS composition contribute to colony morphogenesis remain to be determined.

Pigments excreted by bacteria have long been assumed to be “secondary” metabolites or even waste products, due to the sporadic strain- and condition-dependent nature of their production. (R. P. Williams, Bacteriological Reviews 20, 282 (1956)). Many of these redox-active compounds are known to have antibiotic activities toward competing cells (D. V. Mavrodi, W. Blankenfeldt, L. S. Thomashow, Annu Rev Phytopathol 44, 417 (2006); and K. F. Chater, Philos Trans R Soc Lond B Biol Sci 361, 761 (2006)), but until recently, their potential to directly participate in the physiology of the producing organism has been largely neglected (M. E. Hernandez, D. K. Newman, Cell Mol Life Sci 58, 1562 (2001)). However, small molecules initially characterized as antibiotics allow intercellular communication within bacterial populations (G. Yim, H. H. Wang, J. Davies, Philos Trans R Soc Lond B Biol Sci 362, 1195 (2007)). According to aspects of the disclosure, a conserved function for redox-active pigment antibiotics of the Gram-negative bacterium P. aeruginosa and the Gram-positive bacterium S. coelicolor A3(2) suggests that these functions will be conserved in other bacteria that produce redox-active molecules. These pigments influence transcriptional regulation, and modulate the physical characteristics of communities of their producers at later stages in their development. Rather than being “secondary”, diverse redox-active antibiotics may share similar functions of primary importance throughout the bacterial domain.

### Example 5

### Morphology and Survival of Redox Active Organisms

According to the disclosure colony morphology and survival under anaerobic conditions are connected.

There is a clear morphological difference between a strain (delta phz) that cannot make phenazines (forms flat, wrinkled colonies that cover a large surface area) versus a strain that overproduces phenazines (forms smaller, thicker colonies, covering much less surface area). If a mutant strain that cannot produce phenazines is put in an anaerobic chamber (see electrochemical assay described below), the number of viable cells decreases rapidly over a period of a week unless the strain is “rescued” by providing a small amount (micromolar) of phenazine.

This assay shows that the phenazine is recycled multiple times by the organism (how many times can be calculated by knowing how many cells are in the vessel, the amount of phenazine provided, and the current measured at the electrode). Based on these findings, connection between colony morphology and anaerobic survival as a function of phenazine production can be established. On this basis an electrochemical survival assay was performed according to the following procedure.

Microbiological methods. P. aeruginosa strain UCBPP-PA14 with deletions of two redundant phenazine biosynthetic operons phzA1-G1 and phzA2-G2, hereafter referred to as the Δphz mutant, was cultivated aerobically at 37° C. in Luria-Bertani (LB) broth (Fisher Scientific) to stationary phase. A washing procedure using centrifugation to collect cell pellets and vortexing to resuspend cell pellets in sterile PBS buffer (recipe) was repeated twice. For centrifugation, the cultures were split into 50 mL sterile centrifuge tubes and centrifuged for 8 minutes at 6000 g, at 4° C. After washing, cell culture was concentrated by resuspending pellets in PBS buffer with one-tenth of original cultural volume. Optical density of this culture was measured at 500 nm using Beckman Coulter DU 800 spectrophotometer. Dilution was performed to yield the OD reading below 0.8. The culture aliquots were then transferred into electrochemical cells with oxygen-free minimal salt medium (20 mM glucose as the carbon source) to serve as the cultures electrochemical studies (see next section for details).

Survival experiments in electrochemical setup. Electrochemical measurements were performed with a Gamry G-potentiostat. All experiments were conducted in an O2- and H2-free glovebox (MBraun) at 30° C. and in the dark. All measured potentials were referenced to a Ag/AgCl electrode (RE-5B, BASi). Reported potentials were referenced to the Normal Hydrogen Electrode NHE (V vs. NHE=V vs. Ag/AgCl+207 mV) to permit comparisons with other studies. A graphite rod (6.15 mm dia×152 mm long, Alfa Aesar) was used as the working electrode, and the operating surface area was 6 cm². The counter electrode was made from a platinum mesh (Alfa Aesar) soldered to a copper wire. The working electrode was in a sample compartment together with the reference electrode. The counter electrode was in another compartment isolated from the working electrode. The two compartments were joined by a fritted glass junction. The controlled potential bulk electrolysis was set at 0 V vs. Ag/AgCl. This potential is mild enough to only oxidize reduced phenazines, but not other components in the cultural medium.

To test whether phenazine electron shuttling can help P. aeruginosa survive, 100 μM phenazine, 20 mM glucose, high or low initial cell densities in the range of 109 cells/mL or 107 cells/mL were added to the medium to give total 100 mL suspension in the compartment along with the working and reference electrodes, and the potential was applied at 0 V (vs. Ag/AgCl) for 7 days. Cell suspension aliquots were taken as a function of time for viable colony counts. Current and charge transferred were recorded continuously. Cultures without phenazine, or without the potential applied, or without both were served as the controls. To confirm that glucose is used as the carbon source in the case of survival, experiments with or without glucose addition were compared.

### Example 6

### Redox-Active Molecule Promote Survival of Redox Active Organisms Under Anaerobic Conditions

To investigate the effect of redox-active small molecules on anaerobic survival of redox active organisms, Applicants tested the effect of redox-active molecule in P. aeruginosa PA14. In particular, Applicants assembled bulk electrolysis-based glass bioreactors housed within an O₂- and H₂-free glovebox (MBraun) and controlled by a multichannel potentiostat (Gamry, Series G 300) outside the glovebox. Each bioreactor held a graphite rod working electrode (Alfa Aesar) with an operating surface area of 6 cm², a Ag/AgCl reference electrode (BASi, RE-5B) with a constant potential of +0.207 V vs. the normal hydrogen electrode (NHE), and about 100 mL MOPS culture medium [100 mM MOPS at pH 7.2, 93 mM NH₄Cl, 43 mM NaCl, 2.2 mM KH₂PO₄, 1 mM MgSO₄, 5.0 μM FeCl₃] (modified from ref. (Palmer, K. L., L. M. Mashburn, P. K. Singh, and M. Whiteley. 2005. Cystic fibrosis sputum supports growth and cues key aspects of Pseudomonas aeruginosa physiology. Journal of Bacteriology 187:5267-5277)).

The bioreactor was joined by a fritted glass junction to a small side chamber, in which a Pt counter electrode made from Pt mesh (Alfa Aesar) soldered to a copper wire was held to make the system a complete circuit. In order to selectively examine different redox-active small molecules, Applicants used the phenazine-null mutant (Δphz) of PA14 with deletions of phenazine biosynthetic operons (Dietrich, L. E. P., A. Price-Whelan, A. Petersen, M. Whiteley, and D. K. Newman. 2006. The phenazine pyocyanin is a terminal signalling factor in the quorum sensing network of Pseudomonas aeruginosa. Molecular Microbiology 61:1308-1321) for this study.

Applicants started out by focusing on three endogenous phenazines-pyocyanin (PYO), phenazine-1-carboxylic acid (PCA), and 1-hydroxyphenazine (1-OHPHZ)—that are known to be excreted by PA14 during the stationary-phase growth cycle in laboratory cultures (Dietrich, L. E. P., A. Price-Whelan, A. Petersen, M. Whiteley, and D. K. Newman. 2006. The phenazine pyocyanin is a terminal signalling factor in the quorum sensing network of Pseudomonas aeruginosa. Molecular Microbiology 61:1308-1321.). Applicants subcultured the concentrated cells from aerobic growth to stationary phase in Luria-Bertani (LB, Fisher Scientific) medium at 37° C. into the bioreactor for anaerobic survival and growth tests over a period of seven days at 30° C. To perform the survival experiments, Applicants incubated dense suspensions (10⁹ cells/mL) in the MOPS media containing 20 mM D-glucose to ensure excess electron donor, added about 90 μM phenazine (PYO, or PCA, or 1-OHPHZ), and poised the working electrode at +0.2 V vs. NHE to make certain it was just high enough to efficiently oxidize bacterially reduced phenazine but not other medium components (e.g., D-glucose). Throughout the incubation period, Applicants continuously recorded the anodic (oxidation) currents as well as the charge transferred due to the oxidation of electrochemically active component(s) at the working electrode, and periodically sampled cell suspension aliquots for viable cell counts as measured by colony forming units (CFU) on LB agar (Eschbach, M., K. Schreiber, K. Trunk, J. Buer, D. Jahn, and M. Schobert. 2004. Long-term anaerobic survival of the opportunistic pathogen Pseudomonas aeruginosa via pyruvate fermentation. Journal of Bacteriology 186:4596-4604.).

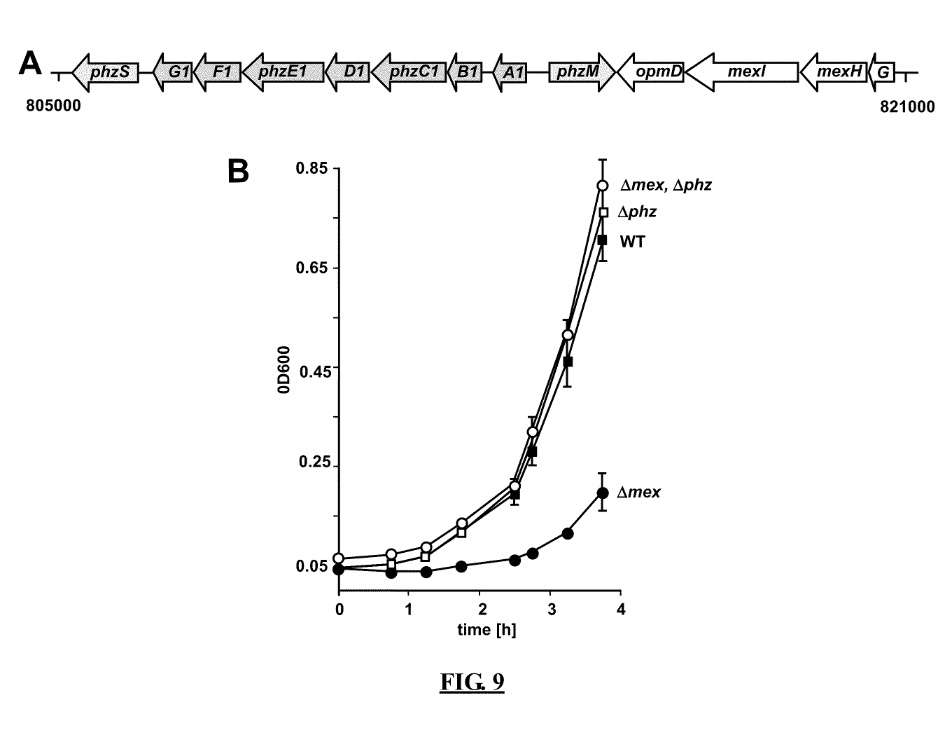

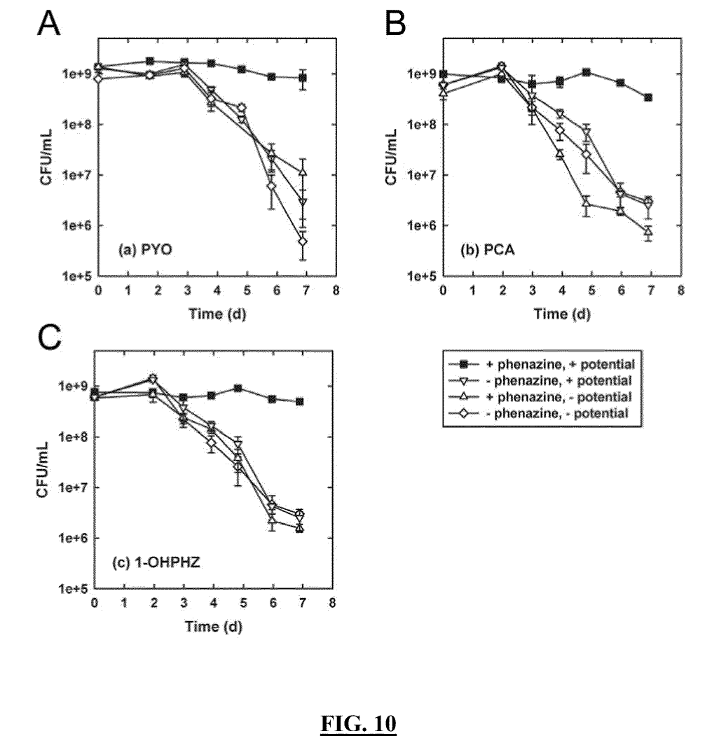

The results are summarized in the illustration of FIG. 10. In particular, Applicants observed that the Δphz mutant maintained a constant viable cell number at the original 10⁹ CFU/mL over seven days, characteristic of survival but not growth (FIG. 10). In contrast, when Applicants incubated the Δphz mutant in the bioreactors without adding phenazine or applying a potential, or both, the viable cells only sustained well for no more than 3 days, and then dropped logarithmically down to 0.1-1% of the original 10⁹ CFU/mL by day 7 (FIG. 10).

The electrochemical observations were in agreement with the CFU results. Without phenazine supplemented in media, Applicants observed a constant anodic current in the range of 5-10 μA with the poised potential, reflecting a background current due to slow oxidation of medium components or direct electron transfer from cells to the working electrode, which was not able to help Δphz survive over 7 days. In the presence of phenazine, on the other hand, the anodic currents increased from the background level to 80±10 μA for PYO and PCA, 45±10 μA for 1-OHPHZ within 2 hours, and stayed at the high current levels with slow decay (less than 20%) throughout the incubation period. The slow current decay was likely due to the electrode fouling and/or the accumulation of toxic metabolic byproducts in the batch reactors over time. Obviously, the facile reversibility of redox-active phenazine, which was reduced within the bacterial cell and oxidized outside the cell by the working electrode as the sole electron acceptor, led to the high current readings and was key to Δphz survival.

Applicants then estimated the average number of phenazine redox cycles (a) based on the equation (Eq. 1) adapted from the Faraday's law for bulk electrolysis (Bard, A. J., and L. R. Faulkner. 2001. Electrochemical Methods: Fundamentals and Applications. John Wiley, New York.):

Q=2FN=2F(acv) [1]

with c as phenazine concentration (M), v as reaction volume (L), and F as Faraday's constant (96485 C/mole), N (=acv) as the amount of phenazine (in moles) involved in the electrolysis, Q (in unit of C) as the net charge quantity associated with the electrochemical oxidation of reduced phenazine during the electrolysis (by subtracting the background charge without phenazine from the total charge passed with phenazine). By recording Q, and knowing c and v, we calculated the number of redox cycles over 7 days for PYO, PCA, and 1-OHPHZ to be 31, 22, and 14, respectively.

Moreover, each of the three phenazines showed the color characteristic of its oxidized form during redox cycling rather than its reduced form when the cycling was not allowed by not applying the poised potential (Wang, Y., and D. K. Newman. 2008. Redox reactions of phenazine antibiotics with ferric (hydr)oxides and molecular oxygen. Environmental Science & Technology 42:2380-2386), indicating that the intracellular phenazine reduction was the rate-limiting step of each redox cycle. In addition, Applicants observed a correlation between the reaction kinetics and phenazine thermodynamic properties: both phenazine reduction potential (see Table 2 below) and the intracellular reduction rate decreased in the order: PYO>PCA>1-OHPHZ. In summary, despite different electron shuttling efficiencies, all three phenazines supported Δphz survival equally well within the testing period by acting as electron acceptors (FIG. 10).

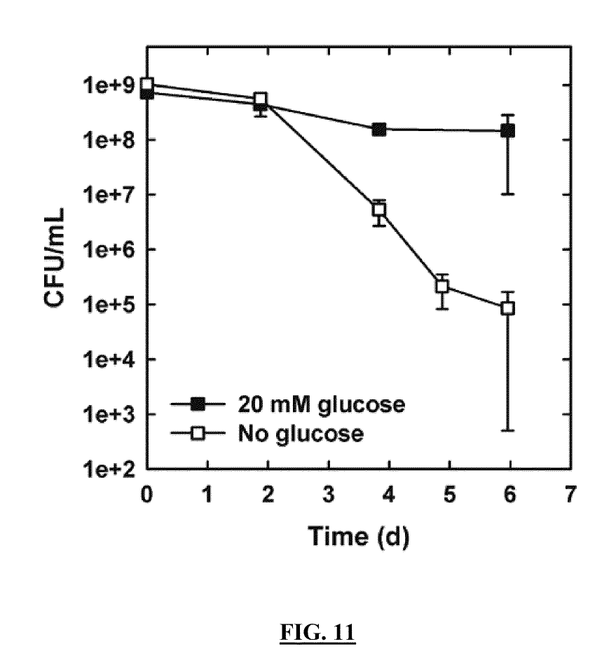

By comparing the survival of Δphz in media with or without D-glucose in pair wise experiments, Applicants confirmed that D-glucose was the electron donor for phenazine electron shuttling supported survival. As shown in FIG. 11, without D-glucose, with the supplement of PYO and the poised potential, Δphz maintained a constant viable cell number at 10⁹ CFU/mL for 2 days and then dropped by 4 orders of magnitude of the original CFU by day 6. The results also indicated that the survival over the first 2-3 days was independent of phenazine electron shuttling and glucose utilization. As being observed in other studies, bacterial cells are able to store some internal energy and carbon sources to support their short-term survival. Applicants investigated whether the phenazine electron shuttling supported survival was specific for the dense suspensions (10⁹ CFU/mL), where too little energy was allocated to each individual cell to stimulate growth. To address this question, Applicants performed the bioreactor experiments as above mentioned with viable cells at 10⁷ CFU/mL, about 1% CFU of the dense suspensions, we observed exactly the same results as for dense suspensions, i.e., PYO electron shuttling supported only survival but not growth (data not shown).

To determine whether the observed electron shuttling promoted P. aeruginosa survival was unique to endogenous phenazines or more generalizable, Applicants performed analogous bioreactor experiments with four other redox-active compounds listed in Table 2.

TABLE 2

| Chemical | | E⁰’ | # of Re- | | |
|---|---|---|---|---|---|
| name | | (vs. | dox cycles | | |
| (Abbrevi- | Structure | NHE) | over 7 | Support | Reduction |
| ation) | (The oxidized form) | (mV) | days | survival? | by PA14? |
| Pyocyanin (PYO) | 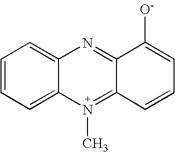 | −40^{a} | 31 | Yes | Yes |
| Phenazine-1- carboxylate (PCA) | 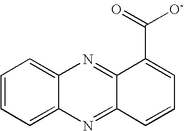 | −114^{a} | 22 | Yes | Yes |
| 1-Hydroxy- phenazine (1-OHPHZ) | 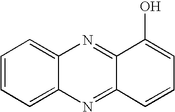 | −174^{a} | 14 | Yes | Yes |
| Methylene blue (MB) | 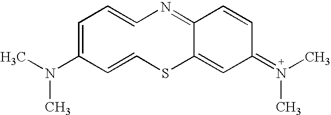 | 0^{b} (+11^{c}) | 3 | No | Yes |
| 2,6-AQDS | 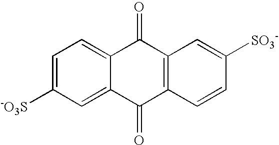 | −184^{d} | No cycle | No | Yes (very slowly) |
| Paraquat | 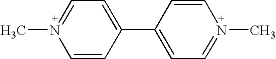 | −446^{e} | No cycle | No | No |
| Homogentisic acid (HMA) | 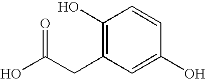 | +306^{b} | No cycle | No | — |

^{a}Reference (Wang, Y., and D. K. Newman. 2008. Redox reactions of phenazine antibiotics with ferric (hydr)oxides and molecular oxygen. Environmental Science & Technology 42:2380-2386)
^{b}E⁰’ values were measured in aqueous solution at pH 7 in this study
^{c}Reference (Fultz, M. L., and R. A. Durst. 1982. Mediator Compounds for the Electrochemical Study of Biological Redox Systems-a Compilation. Analytica Chimica Acta 140:1-18)
^{d}Reference (Hernandez, M. E., and D. K. Newman. 2001. Extracellular electron transfer. Cellular and Molecular Life Sciences 58:1562-1571)
^{e}References (Michaelis, L., and E. S. Hill. 1933. Potentiometric Studies On Semiquinones. Journal of the American Chemical Society 55:1481-1494, Michaelis, L., and E. S. Hill. 1933. The Viologen Indicators. The Journal Of General Physiology 16:859-873)

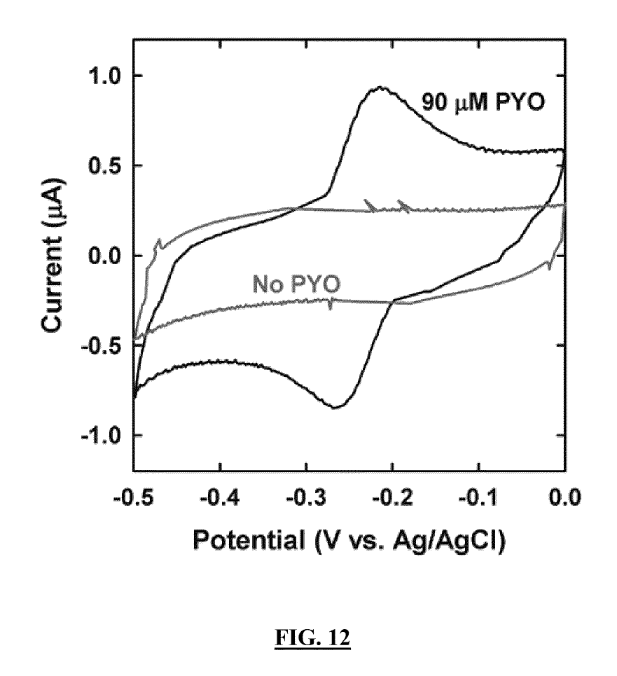

MB is a synthetic compound that shares the core redox-active structure of natural phenazines. Its cyclic voltammetry (CV) at pH 7 exhibited reversible voltammetry peaks centered at 0 mV (vs. NHE) (Table 2), about 40 mV higher than the phenazine PYO (FIG. 12), indicating that MB was electrochemically redox active. In the survival control experiments without a poised potential, the color of MB changed from its oxidized form (blue) to its reduced form (colorless), confirming that MB was reduced intracellularly. In contrast, in the survival experiments with the poised potential, MB stayed the blue color, suggesting that the reduced MB can be readily oxidized by the electrode surface. Unlike PYO, the redox cycling of MB was so inefficient that the current (˜12 μA) with MB was only marginally higher than the background current (5-10 μA), and the calculation showed that MB oxidation-reduction only cycled 3 times over 7 days. The viable cells dropped 3 orders of magnitude regardless of bioreactor experimental conditions, revealing that MB redox cycling cannot support Δphz survival. 2,6-AQDS, the well-studied anthraquinone type exogenous electron shuttle for Shewanella and Geobacter species (Lovley, D. R., J. D. Coates, E. L. BluntHarris, E. J. P. Phillips, and J. C. Woodward. 1996. Humic substances as electron acceptors for microbial respiration. Nature 382:445-448; Newman, D. K., and R. Kolter. 2000. A role for excreted quinones in extracellular electron transfer. Nature 405:94-97), has a reduction potential similar to the phenazine 1-OHPHZ (Table 2). In contrast to 1-OHPHZ, Applicants did not observe 2,6-AQDS redox cycling between the electrode surface and Δphz cells, due to a slow intracellular 2,6-AQDS reduction backed up by the following observation. For the survival control experiments without a poised potential, after 7 days' incubation, the cell cultures showed a faint orange color, indicating that only a small portion of 2,6-AQDS was reduced considering the oxidized 2,6-AQDS is colorless and the reduced is bright red-orange in the 100 μM concentration range (Newman, D. K., and R. Kolter. 2000. A role for excreted quinones in extracellular electron transfer. Nature 405:94-97). Not surprisingly, 2,6-AQDS was not able to support Δphz survival as measured by the viable cell numbers described previously.

Paraquat is a redox-active compound that undergoes reversible single electron transfer between the colorless oxidized form and the blue-colored reduced radical with a reduction potential (−446 mV vs. NHE, pH 7) lower than most cellular reducing equivalents (e.g., NAD(P)H, reduced glutathione) (Michaelis, L., and E. S. Hill. 1933. Potentiometric Studies On Semiquinones. Journal of the American Chemical Society 55:1481-1494, Michaelis, L., and E. S. Hill. 1933. The Viologen Indicators. The Journal Of General Physiology 16:859-873). Despite its low reduction potential, paraquat is known for its ability to undergo in vivo redox cycling in some eukaryotic and bacterial cells (Bus, J. S., and J. E. Gibson. 1984. Paraquat—Model For Oxidant-Initiated Toxicity Environmental Health Perspectives 55:37-46). The reduced paraquat radical produced during this process can react with intracellular oxygen and catalyze the formation of toxic superoxide radical (Bus, J. S., and J. E. Gibson. 1984. Paraquat—Model For Oxidant-Initiated Toxicity Environmental Health Perspectives 55:37-46), mechanistically similar to that of PYO induced toxicity under aerobic conditions (Hassan, H. M., and I. Fridovich. 1980. Mechanism Of The Antibiotic Action Of Pyocyanine. Journal of Bacteriology 141:156-163; Hassett, D. J., L. Charniga, K. Bean, D. E. Ohman, and M. S. Cohen. 1992. Response Of Pseudomonas-Aeruginosa To Pyocyanin—Mechanisms Of Resistance, Antioxidant Defenses, And Demonstration Of A Manganese-Cofactored Superoxide-Dismutase. Infection and Immunity 60:328-336; Mahajan-Miklos, S., M. W. Tan, L. G. Rahme, and F. M. Ausubel. 1999. Molecular mechanisms of bacterial virulence elucidated using a Pseudomonas aeruginosa Caenorhabditis elegans pathogenesis model. Cell 96:47-56). In contrast to PYO, we did not observe paraquat electron shuttling between the electrode surface and Δphz cells, because it cannot be reduced by Δphz in the first place. We did not observe any reduction-associated color change or current readings higher than the background level. Subsequently, paraquat cannot support anaerobic survival of Δphz according to the CFU measurements. Homogentisic acid (HMA) is a phenolic small molecule known as the primary precursor for synthesizing microbial (pyo)melanin (3, 18). For (pyo)melanin-producing organisms, including some P. aeruginosa strains, HMA is secreted in its reduced form, auto-oxidized, and polymerized into red-brown humic-like compound, (pyo)melanin (Chatfield, C. H., and N. P. Cianciotto. 2007. Secreted pyomelanin pigment of Legionella pneumophila confers ferric reductase activity. Infection and Immunity 75:4062-4070; Nosanchuk, J. D., and A. Casadevall. 2003. The contribution of melanin to microbial pathogenesis. Cellular Microbiology 5:203-223; Turick, C. E., L. S. Tisa, and F. Caccavo. 2002. Melanin production and use as a soluble electron shuttle for Fe(III) oxide reduction and as a terminal electron acceptor by Shewanella algae BrY. Applied and Environmental Microbiology 68:2436-2444), which has been reported to function as an electron shuttle for enhanced Fe(III) reduction in Shewanella species (Turick, C. E., L. S. Tisa, and F. Caccavo. 2002. Melanin production and use as a soluble electron shuttle for Fe(III) oxide reduction and as a terminal electron acceptor by Shewanella algae BrY. Applied and Environmental Microbiology 68:2436-2444). By performing cyclic voltammetry (CV) and the CV peak analysis as described previously (Wang, Y., and D. K. Newman. 2008. Redox reactions of phenazine antibiotics with ferric (hydr)oxides and molecular oxygen. Environmental Science & Technology 42:2380-2386), we determined that HMA is subject to reversible oxidation-reduction via single electron transfer, resulting in a reduction potential of +306 mV vs. NHE (pH 7), higher than the potential applied to test for Δphz survival. This led to the oxidation of HMA by the electrode was not thermodynamically feasible. As expected, HMA cannot support Δphz survival. In summary, our results imply that electron shuttling promoted P. aeruginosa survival is likely to be specific to endogenous phenazines, not other type redox-active molecules.

The fact that phenazine electron shuttling can only support the survival but not the growth of Δphz is probably because the phenazine reduction energy is decoupled from the respiratory chain necessary for growth, or the amount of energy generated through the respiratory chain is insufficient to support growth. Phenazines are redox-active small molecules produced by Pseudomonas species, which often form biofilms on a variety of surfaces, including the lungs of cystic fibrosis patients and the electrodes of microbial fuel cells. Beyond merely serving as antibiotics, recent studies suggest that phenazines may be important in biofilm physiology through their ability to shuttle electrons between bacteria and not easily accessible terminal oxidants (e.g., insoluble Fe(III) minerals and diffusion-limited O₂).

In conclusion, our findings indicate that altering microbial access to/usage of small molecule electron shuttles may directly impact their survival: under conditions where these organisms are pathogens (e.g. in infections), disrupting their usage could improve means to inhibit their growth; under conditions where these organisms are beneficial, stimulating their ability to cycle these compounds (e.g. in microbial fuel cells) could improve their performance.

Having thus described several aspects of at least one embodiment of this disclosure, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the present description and drawings are by way of example only.

The examples set forth above are provided to give those of ordinary skill in the art a complete disclosure and description of how to make and use the embodiments of the, compounds, compositions, systems and methods of the disclosure, and are not intended to limit the scope of what the inventors regard as their disclosure. All patents and publications mentioned in the disclosure are indicative of the levels of skill of those skilled in the art to which the disclosure pertains. The present disclosure also includes an Appendix A that is enclosed with the present disclosure and forms integral part of the same.

The entire disclosure of each document cited (including patents, patent applications, journal articles, abstracts, laboratory manuals, books, or other disclosures) is hereby incorporated herein by reference. In particular all publications, patents and sequence database entries mentioned herein, including those items listed below, are hereby incorporated by reference in their entirety as if each individual publication or patent was specifically and individually indicated to be incorporated by reference. In case of conflict, the present application, including any definitions herein, will control.

It is to be understood that the disclosures are not limited to particular compositions or biological systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The terms “multiple” and “plurality” includes two or more referents unless the content clearly dictates otherwise. Unless defined otherwise, all technical and scientific terms used herein have the same meaning as commonly understood by one of ordinary skill in the art to which the disclosure pertains.

A number of embodiments of the disclosure have been described. Nevertheless, it will be understood that various modifications may be made without departing from the spirit and scope of the present disclosure. In particular, modifications of the above-described modes for carrying out the disclosure that are obvious to persons of skill in the art are intended to be within the scope of the present disclosure. Accordingly, other embodiments are within the scope of the following claims.

1. A method for identifying a compound able to affect production and/or activity of a redox active compound, the method comprising:
(i) contacting a test colony of redox active unicellular organism with a test agent;
(ii) after contacting, determining a morphological parameter of the test colony; and
(iii) comparing the morphological parameter to a reference, wherein the comparison is indicative of whether or not the test agent is a compound able to affect the production and/or activity of a redox active compound in the redox active unicellular organism unicellular organism.

2. The method of claim 1, further comprising acquiring a digital image of the colony, wherein the morphological parameter is determined from the digital image.

3. The method of claim 1, wherein the morphological parameter is selected from: texture (smooth/wrinkled), size (spread/compact), area, perimeter, perimeter to area ratio, rugosity, volume, major axis length, minor axis length, equivalent ellipse area, area to equivalent ellipse area ratio, bounding box length, bounding box width, bounding box length to width ratio, bounding box area, convex hull area, convex hull perimeter, average radius, average fiber length, and average fiber width.

4. A method for identifying a candidate therapeutic compound, comprising:
(i) contacting a test colony of redox active bacteria with a test agent;
(ii) after contacting, determining a morphological parameter of the test colony; and
(iii) comparing the morphological parameter to a reference, wherein the comparison is indicative of whether or not the test agent is a candidate therapeutic compound

5. The method of claim 4, further comprising acquiring a digital image of the colony, wherein the morphological parameter is determined from the digital image.

6. The method of claim 4, wherein the morphological parameter is selected from: texture (smooth/wrinkled), size (spread/compact), area, perimeter, perimeter to area ratio, rugosity, volume, major axis length, minor axis length, equivalent ellipse area, area to equivalent ellipse area ratio, bounding box length, bounding box width, bounding box length to width ratio, bounding box area, convex hull area, convex hull perimeter, average radius, average fiber length, and average fiber width.

7. The method of claim 4, wherein the reference is the value of the morphological parameter in a control colony.

8. The method of claim 7, wherein the control colony is a colony of redox active bacteria that has not been contacted with the test agent.

9. The method of claim 7, wherein the morphological parameter is area, and wherein a statistically significant increase in area of the test colony compared with the control colony indicates that the test agent is a candidate therapeutic compound.

10. The method of claim 7, wherein the morphological parameter is shape, and wherein a statistically significant increase in spread of the test colony compared with the control colony indicates that the test agent is a candidate therapeutic compound.

11. The method of claim 7, wherein the morphological parameter is texture, and wherein a statistically significant increase in the wrinkledness of the test colony compared with the control colony indicates that the test agent is a candidate therapeutic compound.

12. The method of claim 7, wherein the control colony is a colony of redox active bacteria having a mutation in a phenazine biosynthetic gene, and wherein a lack of a statistically significant difference between the morphological parameter and the reference indicates that the test agent is a candidate therapeutic compound.

13. The method of claim 4, wherein the reference is a predetermined value.

14. The method of claim 13, wherein the predetermined value is maximum area and if the maximum area is statistically significantly greater than the predetermined value of about 2.5 cm², then the test agent is identified as a candidate therapeutic compound.

15. The method of claim 13, wherein steps (i) and (ii) are repeated at two or more intervals.

16. The method of claim 4, wherein redox active bacteria is an Actinobacteria or a Proteobacteria.

17. A method of treating a bacterial infection in a subject, the method comprising administering an inhibitor which reduces the bacterial levels of a redox-active compound.

18. The method of claim 17, wherein the bacterial infection derives from the Pseudomonas genus and/or from the Streptomyces genus.

19. The method of claim 17, wherein the redox-active compound is a phenazine.

20. The method of claim 17, wherein the redox-active compound is selected from the group consisting of a quinone, hydroquinone, semiquinone, flavins, anthraquinone and a quinolone.

21. The method of claim 17, wherein the bacterial infection is resistant to antibiotics comprising the beta-lactam antibiotics, which comprise penicillin, piperacillin, imipenem, tobramycin, and ciprofloxacin.

22. The method of claim 17, wherein the inhibitor is administered in combination with an antibiotic.

23. The method of claim 17, where the inhibitor is selected from a plurality of candidate therapeutic compounds obtained according to a method comprising
(i) contacting a test colony of redox active bacteria with a test agent;
(ii) after contacting, determining a morphological parameter of the test colony; and
(iii) comparing the morphological parameter to a reference, wherein the comparison is indicative of whether or not the test agent is a candidate therapeutic compound

24. A pharmaceutical composition comprising an inhibitor of bacterial production of redox active molecule and a pharmaceutically suitable carrier.

25. The pharmaceutical composition of claim 24, wherein the inhibitor is selected from a plurality of candidate therapeutic compounds obtained according to a method comprising
(i) contacting a test colony of redox active bacteria with a test agent;
(ii) after contacting, determining a morphological parameter of the test colony; and
(iii) comparing the morphological parameter to a reference, wherein the comparison is indicative of whether or not the test

26. A method for identifying a candidate compound for enhancing power output of a microbial fuel cell, comprising:
(i) contacting a redox active bacterial colony with a test agent;
(ii) after contacting, determining morphology of the redox active bacterial colony;
(iii) comparing the morphology of the redox active bacterial colony to a reference, wherein the comparison is indicative of whether or not the test agent is a candidate compound for enhancing power output of a microbial fuel cell.

27. The method of claim 26, further comprising acquiring a digital image of the colony, wherein the morphological parameter is determined from the digital image.

28. The method of claim 26, wherein the morphological parameter is selected from: texture (smooth/wrinkled), size (spread/compact), area, perimeter, perimeter to area ratio, volume, major axis length, minor axis length, equivalent ellipse area, area to equivalent ellipse area ratio, bounding box length, bounding box width, bounding box length to width ratio, bounding box area, convex hull area, convex hull perimeter, average radius, average fiber length, average fiber width, volume, and rugosity.

29. The method of claim 26, wherein the reference is the value of the morphological parameter in a control colony.

30. The method of claim 29, wherein the control colony is a colony of redox active bacteria that has been contacted with an exogenous phenazine compound.

31. The method of claim 30, wherein a lack of a statistically significant difference between the morphological parameter and the reference indicates that the test agent is a candidate compound for enhancing power output of a microbial fuel cell.

32. The method of any of claims 29, wherein the morphological parameter is shape, and wherein a statistically significant increase in compactness of the test colony compared with the control colony indicates that the test agent is a candidate compound for enhancing power output of a microbial fuel cell.

33. The method of claim 29, wherein the morphological parameter is texture, and wherein a statistically significant increase in the smoothness of the test colony compared with the control colony indicates that the test agent is a candidate compound for enhancing power output of a microbial fuel cell.

34. The method of claim 26, wherein the reference is a predetermined value.

35. The method of claim 34, wherein the predetermined value is the maximum area and if the maximum area is statistically significantly less than the predetermined value of about 2.5 cm2, then the test agent is identified as a candidate compound for enhancing power output of a microbial fuel cell.

36. The method of claim 35, wherein the redox active bacteria is an Actinobacteria or a Proteobacteria.
